(19) United States
(12) Patent Application Publication
(10) Pub. No.: US 2025/0282706 A1
(43) Pub. Date: Sep. 11, 2025

(54) PROCESS FOR THE MANUFACTURE OF NONAFLUORO-TERT-BUTYL ALCOHOL BY ELECTROFLUORINATION (ECF)

(71) Applicant: FLUORINNOVATION L.L.C-FZ, Nad Al Sheba Dubai (AE)

(72) Inventor: Max Josef Braun, Wedemark (DE)

(73) Assignee: FLUORINNOVATION L.L.C-FZ, Nad Al Sheba Dubai (AE)

(21) Appl. No.: 19/094,226

(22) Filed: Mar. 28, 2025

Related U.S. Application Data
(63) Continuation of application No. PCT/IB2023/059913, filed on Oct. 3, 2023.

(30) Foreign Application Priority Data
Oct. 5, 2022 (DE) 10 2022 125 687.1

(51) Int. Cl. C07C 67/287; C07C 309/65; C25B 3/07; C25B 3/11; C25B 3/28

(52) U.S. Cl. CPC C07C 67/287; C07C 309/65; C25B 3/07; C25B 3/11; C25B 3/28

(57) ABSTRACT

A process for the manufacture of nonafluoro-tert-butyl alcohol includes the use of a hydroxyl group protected nonafluoro-tert-butyl alcohol ester as an intermediate. The hydroxyl group protected nonafluoro-tert-butyl alcohol ester is obtained by subjecting a hydroxyl group protected tert-butyl alcohol ester compound to an electrofluorination (ECF) step. Deprotecting the hydroxyl group protected nonafluoro-tert-butyl alcohol ester results in the unprotected nonafluoro-tert-butyl alcohol. An electrochemical microreactor can be used in the ECF step for the manufacture of the nonafluoro-tert-butyl alcohol compound.

### CROSS REFERENCE TO RELATED APPLICATIONS

This application is a continuation application of international patent application PCT/IB2023/059913, filed on Oct. 3, 2023 and designating the U.S., which claims priority to German patent application 10 2022 125 687.1, filed on Oct. 5, 2022, both of which are hereby incorporated by reference in their entireties.

### TECHNICAL FIELD

The disclosure relates to a new process for the manufacture of nonafluoro-tert-butyl alcohol, commonly also known as perfluoro-t-butanol (perfluoro-t-BuOH).

### BACKGROUND

The compound nonafluoro-tert-butyl alcohol (IUPAC name: 1,1,1,3,3,3-hexafluoro-2-(trifluoromethyl)-propan-2-ol) is a completely fluorinated alcohol. Other names are perfluoro-tert-butyl alcohol, perfluoro-tert-butanol or perfluoro-t-butanol (perfluoro-t-BuOH), respectively.

Typical technical applications of the compound perfluoro-t-butanol (perfluoro-t-BuOH) are described in the scientific or technical literature as well as in the patent literature. Some older applications of perfluoro-t-butanol (perfluoro-t-BuOH) are in so-called “fluorous chemistry reactions,” for example, as disclosed in the Journal of Fluorine Chemistry (2006), 127(11), pp. 1496-1504. The compound perfluoro-t-butanol (perfluoro-t-BuOH) is also described for use in the preparation of pharmaceutical peptides like in WO 2008/034093. Newer technical applications of the compound perfluoro-t-butanol (perfluoro-t-BuOH) in ionic liquids are described, for example, in New Journal of Chemistry (2017), 41(1), pp. 47-50; and use as heat transfer fluid is described, for example, in CN 111792985; and use in polymer applications is described, for example, in JP 2013/006952 and in the journal Macromolecules (Washington, DC, United States) (2016), 49(10), pp. 3706-3715. Newer patent publications like CN 110563764 describe the large scale use of the compound perfluoro-t-butanol (perfluoro-t-BuOH) as flame retardant in battery electrolyte formulations, and as a starting material for stable peroxides as replacement for SF₆ (which has a huge global warming potential value of 23900; SF₆ is currently used as gaseous dielectric medium in very high industrial amounts in high voltage electricity applications, e.g., as circuit breakers and switch gear). The compound perfluoro-t-butanol (perfluoro-t-BuOH) is also described as an additive in electronics, for example, in WO 2019/207020.

The compound perfluoro-t-butanol (perfluoro-t-BuOH) is the perfluorinated analogue of tert-butyl alcohol (t-butanol; t-BuOH). Notably, as a consequence of its electron withdrawing fluorine substituents, it is very acidic for an alcohol, with a pKₐ value (acidity) of 5.4, similar to that of a carboxylic acid. As another consequence of being a perfluorinated compound, it is also one of the lowest boiling alcohols, with a boiling point lower than that of methanol. (CAS number: 2378-02-1) Chemical formula C₄F₉OH Molecular mass: 236.04 g/mol. It is a colourless liquid, miscible with water, boiling point 45° C. (standard state at 25° C. and 100 kPa). Up to now, only for laboratory suitable methods give access to perfluoro-t-butanol (perfluoro-t-BuOH).

The compound perfluoro-t-butanol (perfluoro-t-BuOH) is usually prepared by addition of trichloromethyllithium to hexafluoroacetone, followed by halogen exchange with antimony pentafluoride. The aluminate derived from its alkoxide anion, tetrakis[1,1,1,3,3,3-hexafluoro-2-(trifluoromethyl)propan-2-oxy]aluminate(1-), {Al[(CF₃)₃CO]₄}⁻ is used as a weakly coordinating anion. However, the use of trichloromethyllithium is hazardous because lithium organyl compounds with halogen in the alpha-position tend to be explosive (dihalocarbene formation).

Another applied laboratory method, e.g., is also using hexafluoroacetone as starting material, and is outlined in more detail later herein below.

All related art processes for the manufacture of the compound perfluoro-t-butanol (perfluoro-t-BuOH) currently known in the related art have a number of disadvantages.

Currently the compound perfluoro-t-butanol (perfluoro-t-BuOH) is produced by the general reaction of a “CF₃⁻ anion” with hexafluoroacetone (HFA) in a nucleophilic reaction as outlined in the following reaction scheme:

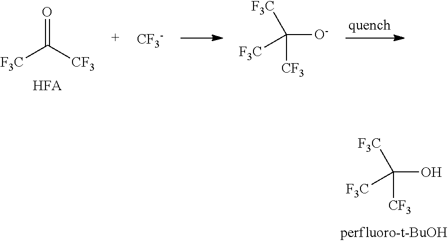

Hexafluoroacetone (HFA) is a gas at room temperature, and therefore besides the involved “chemistry” the costs for HFA usage are sharply increased also by logistic issues, e.g., such like handling and cleaning of pressure cylinders and safety aspects, because HFA is a very toxic gas at atmospheric pressure. Hydrates of HFA are more easy to handle. The most typical hydrate of HFA is HFA trihydrate (HFA×3 H₂O). However, as HFA hydrates have no reactivity vs. trifluoromethyl anions, the HFA hydrates need to be converted to almost anhydrous HFA by means of dehydrating agents like P₂O₅ or SO₃/H₂SO₄. As preparation and handling of suitable HFA is economically and technically the most challenging part of the Pf-t-BuOH synthesis it is outlined in more details below.

In hitherto known industrial scale processes hexafluoroacetone (HFA) usually is made from hexafluoropropylene (HFP) followed by epoxidation to hexafluoropropylene oxide (HFPO) and subsequent isomerization to yield hexafluoroacetone (HFA), as outlined in the following reaction scheme:

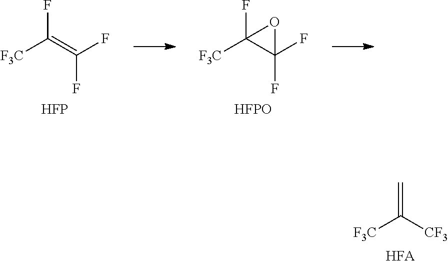

The improved isomerization of HFPO to HFA is disclosed in a recent patent publication, i.e., in the Chinese patent application CN 111116342 A (published May 8, 2020). Also, the isomerization of fluorinated epoxides to related carbonyl compounds is described in an older US patent, namely U.S. Pat. No. 3,321,515 A (May 23, 1967).

The entire synthesis of HFA starting from HFP is disclosed with some improvements, e.g., in the recent Chinese patent application CN 111153783 A (May 15, 2020). Further a device for implementing the process is disclosed. A process for producing hexafluoro-acetone trihydrate by taking hexafluoropropylene as a raw material is disclosed. The process comprises the following steps: (1) taking hexafluoropropylene and oxygen as raw materials, and carrying out oxidation reaction in an oxidation kettle in the presence of a solvent to obtain a mixture of hexafluoropropylene oxide and unreacted hexafluoropropylene; (2) carrying out solvent removal, acid removal, and drying of the obtained mixture, introducing the mixture into a fixed bed reactor, and carrying out isomerization reaction on hexafluoropropylene oxide under the condition of a catalyst to generate hexafluoroacetone; and (3) carrying out multistage water absorption from the obtained product, and combining hexafluoroacetone with water to obtain hexafluoroacetone trihydrate. A mixed product obtained through the oxidation reaction is not separated, hexafluoroacetone and hexafluoroacetone trihydrate are produced directly through a reaction in the presence of a Lewis acid catalyst, separation of hexafluoropropylene and hexafluoroacetone trihydrate is realized according to the boiling point difference, and hexafluoropropylene obtained through separation can be continuously recycled after rectification, drying, and impurity removal. The CN 111153783 A asserts that according to the method, high-difficulty separation of hexafluoropropylene and hexafluoropropylene oxide in the middle step is avoided, the problem that hexafluoropropylene and hexafluoroacetone are difficult to separate in the related art is solved, the production energy consumption and wastewater discharge are reduced, and that costs are saved.

The entire synthesis of HFA is also disclosed in the scientific literature, e.g., by Susumu Misaki in Journal of Fluorine Chemistry, 17 (1981), pp. 159-171 (“Direct Fluorination of Phenol and Cresols”), and by Kurosaki et al. in Chemistry Letters (1988), (1), pp. 17-20.

Even though HFP is produced by several companies like DuPont, Solvay Specialty Polymers, Daikin, and Lianyungang Tetrafluor New Materials Co., Ltd. in large industrial scale and at reasonable price by pyrolysis of (as refrigerant phased out) HCFC-22 (CF₂ClH), HFPO and HFA are quite exotic and expensive compounds due to their gaseous form and toxicity.

For the other component needed in the synthesis of perfluoro-t-butanol (perfluoro-t-BuOH), namely the CF₃⁻ anion, Ruppert's reagent (CF₃-TMS) is a suitable precursor, but there is no availability in large-scale production and at a reasonable price any more. In the past, large-scale production of Ruppert's reagent at a reasonable price was achieved by a process using Halon 1301 (CF₃—Br; used in the 60ies as a very efficient fire extinguishing agent) as a suitable large-scale commercial precursor material. But this suitable precursor for CF₃-TMS (Ruppert's reagent), i.e., the Halon 1301 (CF₃—Br) was phased out during the Montreal Protocol, and thus is not available anymore in large industrial quantities for environmental ban reasons, and not as end product, but it might remain as a niche compound for legally confined applications such as a “a declared use as intermediate only.” An alternative synthesis of Ruppert's reagent, e.g., out of alternative precursor CF₃H, which is still available, for example, is disclosed in WO 2012/148772. Another alternative synthesis of Ruppert's reagent out of expensive CF₃SO₂Cl (triflyl chloride; TfCl), for example, is disclosed in CN 107880069. Said alternative syntheses of Ruppert's reagent still would be possible, but either requires very challenging special equipment, or in case of triflyl chloride (TfCl), the starting material price would be already higher than the acceptable market price of perfluoro-t-butanol (perfluoro-t-BuOH) for larger scale applications like in polymers, SF₆ replacements, and batteries.

Further sources for the CF₃⁻ anion are trifluoroacetates, e.g., such like the sodium or potassium trifluoroacetate, which are available at reasonable price and available in large-scale). In this case, the CF₃⁻ anion is generated by thermally induced decarboxylation of the trifluoroacetate. Earlier publications with that decarboxylation reaction type describe the synthesis, e.g., of CF₃I out of trifluoroacetates (Journal of the American Chemical Society (1950), 72, pp. 3806-7), and of CF₃-substituted benzenes (Chemistry Letters (1981), (12), pp. 1719-20; Journal of Fluorine Chemistry (2010), 131(11), pp. 1108-1112).

It is also known in the related art that the direct contact of alcohols with elemental fluorine (F₂) normally give alkylhypofluorites (see Elemental Fluorine in Organic Chemistry, Springer Verlag 1997, ISBN: 978-3-540-69197-6, DOI doi.org/10.1007/3-540-69197-9), and it is known that alkylhypofluorites can be explosive (J. Fluorine Chemistry 54 (1991), p. 1). For example, hypofluorites are formally derivatives of OF⁻, which is the conjugate base of hypofluorous acid. One example is trifluoromethyl hypofluorite (CF₃OF); trifluoromethyl hypofluorite (CF₃OF) can be regarded as a simple mixture of COF₂ and F₂, and among said alkylhypofluorites it is an exception because it is not explosive, and therefore it can be used as fluorinating agent.

The related art processes for the manufacture of nonafluoro-tert-butyl alcohol, commonly also known as perfluoro-t-butanol (perfluoro-t-BuOH), for example, have the following disadvantages: HFA synthesis involves several complicated steps; HFA is a gas (under standard conditions), and therefore it is difficult to handle HFA. Moreover, HFA is toxic. In addition, convenient and more environmentally friendly processes are desired as compared to syntheses routes using Ruppert's reagent (CF₃-TMS), or using alternative sources of the CF₃⁻ anion, e.g., such process as described above, and the hazards possibly related thereto. Waste waters contaminated with toxic material shall be avoided.

### SUMMARY

It is an object of the present disclosure is to overcome the disadvantages of the related art processes, in particular to provide a more efficient and energy saving processes, and also more environmentally friendly process, for the manufacture of nonafluoro-tert-butyl alcohol, commonly also known as perfluoro-t-butanol (perfluoro-t-BuOH), and providing a convenient synthesis route differently from using hexafluoroacetone (HFA) and other fluorinated building blocks as starting compounds than those presently used in the related art processes.

### BRIEF DESCRIPTION OF DRAWINGS

FIG. 1 depicts the use of TFAC (trifluoroacetylchloride) as protecting group raw material in a coil reactor (continuous process). Reference is made to Example 1.

FIG. 2 depicts the use of TFAC (trifluoroacetylchloride) as protecting group raw material in a counter-current system (batch process) is depicted. Reference is made to Example 2a. Scheme of apparatus and process is shown with counter-current system (batch process), wherein the counter-current column is connected to the reservoir via a pipeline.

FIG. 3 shows, as an example, the electrofluorination (ECF), exemplified by the electrofluorination (ECF) of trifluoroacetic acid tert-butyl ester (batch process) to yield perfluoro-tert-butyl trifluoroacetate. Reference is made to Example 3.

FIG. 4 depicts the use of TFAH (trifluoroacetic acid anhydride) as protecting group raw material in a coil reactor (continuous process). Reference is made to Example 7.

FIG. 5 depicts the continuous preparation of triflate protected t-butanol (t-BuOH), i.e., of tert-butyl trifluorosulfonate ester, in a coil reactor. The raw product reservoir is equipped with a pressure valve going into a basic scrubber (scrubber not shown) for allowing the formed HCl to leave the raw product reservoir. Reference is made to Example 8.

FIG. 6 depicts the use of TFAC (trifluoroacetylchloride) as protecting group raw material in a counter-current system (batch process). Reference is made to Example 2b. Scheme of apparatus and process is shown with counter-current system (batch process), and counter-current column which is directly placed onto the reservoir.

### DESCRIPTION OF EXEMPLARY EMBODIMENTS

The objects of the disclosure are achieved by the process for the synthesis of perfluoro-t-Butanol (Pf-t-BuOH) using electrofluorination (ECF). In particular, the present disclosure relates to a novel process for the manufacture of nonafluoro-tert-butyl alcohol, commonly also known as perfluoro-t-butanol (perfluoro-t-BuOH), involving hydroxyl group protecting groups and an electrofluorination (ECF) step.

As described here above, the compound nonafluoro-tert-butyl alcohol, commonly also known as perfluoro-t-butanol (perfluoro-t-BuOH), is known in the related art, as well as hydroxyl group protected tert-butyl alcohol compound (hydroxyl group protected t-butanol or t-BuOR, respectively, wherein R is a hydroxyl group protecting group).

However, using the hydroxyl group protecting group chemistry in combination with an electrofluorination (ECF) step is not known in the related art, hitherto.

In brief summary, the disclosure relates to a new process for the manufacture of the compound nonafluoro-tert-butyl alcohol, commonly also known as perfluoro-t-butanol (perfluoro-t-BuOH), and of the manufacture of a hydroxyl group protected and methyl group perfluorinated intermediate compound or precursor compound of the targeted compound perfluoro-t-butanol (perfluoro-t-BuOH).

The use of the protective groups, as described herein, for protecting the hydroxyl group in tert-butanol in combination with a subsequent electrofluorination (ECF) step reaction step for perfluorinating the methyl groups of the targeted product compound tert-butanol is novel over the related art. This applies to all of the protective groups mentioned herein in the context of the processes of the disclosure.

The perfluorinated and trifluoroacetyl protected t-butanol compound, i.e., the trifluoroacetic acid tert-butyl ester, is already known from the related art. However, this compound has been made in a different way by the new process of the present disclosure; namely, by reacting perfluoro-t-butanol and trifluoroacetic acid anhydride (TFAH).

The perfluorinated and trifluorosulfonyl (i.e., triflate) protected t-butanol compound, i.e., the trifluorosulfonic acid tert-butyl ester, is also already known from the related art. However, this compound has been made in a different way with the novel process of the present disclosure, namely, by reacting perfluoro-t-butanol and triflic anhydride (Tf₂O).

Accordingly, the manufacture of the hydroxyl group protected and methyl group perfluorinated intermediate compound or precursor compound is novel. In this process according to the disclosure the intermediate compound or precursor compound, i.e., the hydroxyl group protected and methyl group perfluorinated t-butanol (t-BuOH) compound, is obtained by subjecting a hydroxyl group protected t-butanol (t-BuOH) compound to an electrofluorination (ECF) step. The disclosure also relates to a new process for the manufacture of the compound nonafluoro-tert-butyl alcohol, commonly also known as perfluoro-t-butanol (perfluoro-t-BuOH), by deprotecting the hydroxyl group protected and methyl group perfluorinated t-butanol (t-BuOH) compound to obtain the unprotected nonafluoro-tert-butyl alcohol compound, i.e., in other terms the unprotected methyl group perfluorinated perfluoro-t-butanol (perfluoro-t-BuOH) compound.

Deprotecting, i.e., removing the hydroxyl group protecting group may be achieved by conventional manners known to the person skilled in the art. For example, such deprotecting may be achieved by saponification with an aqueous inorganic base, such as sodium hydroxide or potassium hydroxide dissolved in water, and phase separation of the targeted deprotected compound. Due to cost reasons, a simple inorganic base (KOH, NaOH) is the most typical option for deprotection, but other deprotection options like a transesterification, treatment with NaBH₄/LiAlH₄ or even a deprotection by hydrogenation with hydrogen (H₂) over metal catalysts as well as by use of water soluble amines (e.g., NEt₃ with 112 g/l at 20° C.) are different options and technically applicable.

Next to the triethyl amine (NEt₃), depending on their degree of solubility in water, also other water soluble organic amines can be used for deprotecting of the protected hydroxyl group. Thus, water soluble amines suitable for deprotecting reaction in the context of the disclosure, for example, are alkyl amines such as alkyl amines with independently one to three C1- to C3-alkyl chains (e.g., methyl, ethyl, propyl, iso-propyl, and combinations thereof). Accordingly, the C1- to C3-alkyl chain bearing alkyl amines are selected from the group consisting of methylamine (MeNH₂), dimethylamine (Me₂NH), trimethylamine (Me₃N), ethylamine (EtNH₂), diethylamine (Et₂NH), triethylamine (Et₃NH), propylamine (PrNH₂), dipropylamine (Pr₂NH), iso-propylamine (i-PrNH₂), and di-iso-propyl-amine (i-Pr₂NH). In principle, aromatic amines, such as hydroxyl-substituted amines, e.g., m-hydroyaniline (solubility of 26 g/l), are also suitable for deprotecting reaction in the context of the disclosure but besides water solubility less typical than aliphatic amines also due to economic reasons.

Regarding the new process of the disclosure for the synthesis of the compound nonafluoro-tert-butyl alcohol, commonly also known as perfluoro-t-butanol (perfluoro-t-BuOH), surprisingly, now it has been found by the present disclosure, that if the hydroxyl-function (OH-function) in t-butanol firstly is reacted with a protecting group and then the protected t-butanol (t-BuOH) compound is subjected to electrofluorination conditions, the compound perfluoro-t-butanol (perfluoro-t-BuOH), can be obtained by a convenient process also suitable for industrial scale, and allowing for manufacturing of the compound perfluoro-t-butanol (perfluoro-t-BuOH) for price sensitive mass product applications. Accordingly, this convenient process is achieved by protecting the hydroxyl-function (OH-function) in t-butanol followed by deprotecting of the hydroxyl-function (OH-function) after having performed a fluorination step under electrofluorination conditions, in particular as described herein.

The overall general inventive synthesis route, involving in a step an electrolytic cell, is outlined in the reaction scheme below, wherein independently R and X both have the meaning as defined further below:

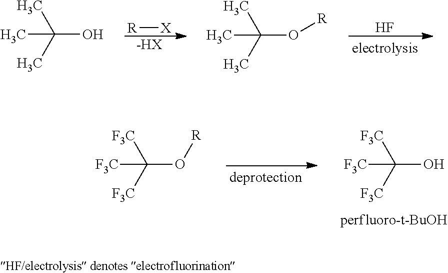

Thus, according to the process of the present disclosure the hydroxyl function (OH-function) in t-butanol firstly is reacted with a protecting group and then is subjected to electrofluorination conditions. Thereby the targeted product perfluoro-t-BuOH can be achieved in a procedure suitable for industrial scale and for allowing price sensitive mass product applications, and in a convenient manner. This is achieved by protecting the hydroxyl function (OH-function) followed by deprotection after the fluorination in anhydrous HF under electrofluorination conditions.

In the context of the present disclosure the term “fluorination in hydrogen fluoride,” “fluorination in HF,” “electrolysis in hydrogen fluoride” or “electrolysis in HF” (e.g., “HF/electrolysis” or similar terms) means or is used, respectively, synonymously with the term “electrofluorination” or “electrochemical fluorination,” respectively. Electrofluorination or electrolysis, respectively, in hydrogen fluoride (HF) can take place in hydrogen fluoride (HF) solution or suspension.

The general definition of “electrochemical fluorination” is normally like this: A substrate that is dissolved or suspended in a fluoride-containing solvent is fluorinated by anodic oxidation (see, e.g., Hollitzer and Sartori, Chem. Ing. Tech. 58 (1986), p. 31). The substrate according to the present disclosure is a hydroxyl group protected tert-butyl alcohol compound (hydroxyl group protected t-butanol or t-BuOR, respectively, wherein R is a hydroxyl group protecting group as defined herein).

In the context of the electrofluorination of the present disclosure the term “HF” or “hydrogen fluoride,” respectively, is meant to denote always “anhydrous hydrogen fluoride” (“AHF”), unless expressively stated otherwise.

Electrofluorination conditions can be provided by any electrochemical (electrolytic) apparatus suitable for performing an electrolysis reaction in anhydrous hydrogen fluoride (“AHF”), i.e., any apparatus resistant to hydrogen fluoride (Hf) and allowing for applying an electric current and voltage to perform an electrochemical (electrolytic) reaction.

General guidance on applicable electrofluorination conditions is given, for example, by Hollitzer and Sartori, in Chem. Ing. Tech. 58 (1986) pp. 31-38 (a review on electrochemical fluorination in hydrogen fluoride solution or suspension), by Pletcher et al., in Chem. Rev. 2018, 118, 9, pp. 4573-4591 (article on flow electrolysis cells for the synthetic organic chemistry laboratory), and by Winterson et. al in Chem. Sci., 2021, 12, p. 9053 (article on electrochemical reactions in flow chemistry involving amine×HF adducts as fluorine (“F”) source).

The electrochemical (electrolytic) apparatus for providing the required electrofluorination conditions, for example, can be an electrolytic cell or electrolysis cell, respectively, in particular the so-called Simmons electrolysis cell, or the electrochemical (electrolytic) apparatus for providing the required electrofluorination conditions, for example, can be an electrochemical (electrolytic) microreactor or electrolysis microreactor, respectively, in particular a novel innovative electrochemical microreactor concept developed by Fraunhofer IMM, an electrochemical microreactor concept addressing especially the aspects modularity, flexibility, high pressure operation, and accessibility of production scale.

Such equipment as a continuously operated electrochemical microreactor is unknown for applications in electrofluorination (ECF), and thus, the present disclosure also pertains to a process comprising an electrofluorination (ECF) process step, wherein a hydroxyl group protected tert-butyl alcohol compound (hydroxyl group protected t-butanol or t-BuOR, respectively, wherein R is a hydroxyl group protecting group as defined herein) is subjected to a fluorination step under electrofluorination conditions in an electrochemical microreactor, in particular as described herein. Further, the present disclosure also pertains to a process wherein the targeted nonafluoro-tert-butyl alcohol, commonly also known as perfluoro-t-butanol (perfluoro-t-BuOH), is manufactured by a process comprising the steps of protecting the hydroxyl-function (OH-function) in t-butanol followed by a deprotecting step of the hydroxyl-function (OH-function) after having performed a fluorination step under electrofluorination conditions in an electrochemical microreactor, in particular as described herein.

In the overall general inventive synthesis route in the reaction scheme here above, the group X of the protecting reagent denotes (e.g., as a leaving group), a hydrogen atom, a halogen atom (typically a fluorine atom or a chlorine atom, more typically a chlorine atom) or an —O—R group (i.e., forming with the R of R—X an anhydride group R—O—R). Typically, the group X of the protecting reagent denotes a hydrogen atom, a chlorine atom or an —O—R group (i.e., forming with the R of R—X an anhydride group R—O—R).

In the overall general inventive synthesis route in the reaction scheme here before, the substituent R of the protecting reagent and the protecting group, respectively, denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C₂-C₄ residue.

More typically the substituent R of the protecting reagent and the protecting group, respectively, denotes a substituent selected from the group consisting of CF₃CO—, CF₂HCO—, CF₂ClCO—, CH₃CO—, CF₃SO₂—, CH₃SO₂—, PfCO—, PfSO₂—, and wherein Pf denotes a partially or perfluorinated C₂-C₄ residue.

Even more typically the substituent R of the protecting reagent and the protecting group, respectively, denotes a substituent selected from the group consisting of CF₃CO—, CF₂HCO—, CF₂ClCO—, CH₃CO—, CF₃SO₂—, and CH₃SO₂—.

Most typically the substituent R of the protecting reagent and the protecting group, respectively, denotes a substituent selected from the group consisting of CF₃CO—, CF₂HCO—, CF₂ClCO—, and CF₃SO₂—.

Particular and representative examples of the above defined protecting reagents R—X, and related protecting groups R, respectively, used in the process of manufacturing nonafluoro-tert-butyl alcohol (perfluoro-t-butanol; perfluoro-t-BuOH), and of the intermediate or precursor compound nonafluoro-tert-butyl alcohol ester (perfluoro-t-butanol ester; perfluoro-t-BuOR), by electrofluorination (ECF) are selected from the group consisting of: trifluoroacetic acid anhydride (TFAH) and trifluoroacetyl chloride (TFAC), both, TFAH and TFAC for protecting the hydroxyl group of the staring material compound tert-butyl alcohol (t-butanol; t-BuOH) by the trifluoroacetyl group; and triflic anhydride (Tf₂O) and triflyl chloride (TfCl), both, Tf₂O and TfCl for protecting the hydroxyl group of the staring material compound tert-butyl alcohol (t-butanol; t-BuOH) by the triflyl group CF₃SO₂—) forming together with the hydroxyl group oxygen a triflate group.

Particular typical protecting reagents R—X used in the process of the present disclosure are TFAH, TFAC, and triflate (CF₃SO₂—) protecting group. More typically, the protecting reagent R—X used in the process of the present disclosure is TFAC and triflate (CF₃SO₂—) protecting group, and most typically the protecting reagent R—X used in the process of the present disclosure is TFAC and TfCl (triflyl chloride). Most typically, the protecting reagent R—X used in the process of the present disclosure is TFAC.

The triflyl group, more formally known as trifluoromethanesulfonyl group, is a functional group with the formula F₃CSO₂—. The triflyl group is often represented by -Tf The related triflate group (trifluoromethanesulfonate) has the formula CF₃SO₂O—, and is represented by —OTf. Triflic anhydride (CF₃SO₂)₂O is known to be a very strong triflating agent.

Triflic acid, the short name for trifluoromethanesulfonic acid, TFMS, TFSA, HOTf or TfOH, is a sulfonic acid with the chemical formula CF₃SO₃H. It is one of the strongest known acids. Triflic acid is mainly used in research as a catalyst for esterification.

Trifluoromethanesulfonic anhydride, also known as triflic anhydride, is the chemical compound with the formula (CF₃SO₂)₂O. It is the acid anhydride derived from triflic acid. This compound is often used as a strong electrophile for introducing the triflyl group, CF₃SO₂. Abbreviated as Tf₂O, triflic anhydride is the acid anhydride of the strong acid triflic acid, CF₃SO₂OH.

Trifluoromethanesulfonyl chloride (or triflyl chloride, CF₃SO₂Cl), abbreviated (TfCl), can be used in a highly efficient method to introduce a trifluoromethyl group to aromatic and heteroaromatic systems. The chemistry is general and mild, and uses a photoredox catalyst and a light source at room temperature.

The protecting group typically has to be selected out of such protecting groups which are not sensitive to elemental fluorine (F₂) and hydrogen fluoride (HF). Accordingly, the usually commonly used TMS-group is not suitable for the process of the present disclosure.

However, some protecting groups which in principle are sensitive to a F₂-fluorination, such as alkyl-, benzyl-, t-butyl-, allyl-, trityl-, tetrahydropyranyl-, methoxybenzyl-, methoxymethyl-, but in principle only, if desired for any other reason, can be used, but in this case at least partial fluorination of said protecting group fragment has to be expected, and after deprotecting recycling of the partially fluorinated protecting groups is very difficult or impossible, a disadvantage in addition to the higher F₂ consumption, anyway. Hence, the use of such fluorinable protecting groups is very inconvenient due to environmental and cost reasons.

Now, according to the present disclosure, for example, the trifluoroacetyl group as protecting group was identified to fulfill all requirements to be used as a protecting group; even though some literature references disclose as the best synthesis route the electrofluorination of acetylchloride (or acetylfluoride) to trifluoroacetylfluoride followed by hydrolysis to obtain to trifluoroacetic acid (Eidman, K. F.; Nichols, P. J. “Trifluoroacetic Acid” in Encyclopedia of Reagents for Organic Synthesis (Ed: L. Paquette) 2004, J. Wiley & Sons, New York. doi:10.1002/047084289). It must be noted that, even while known as a protecting group in general, the trifluoroacetyl group has never been used in electrofluorinations in the presence of anhydrous hydrogen fluoride (AHF), and never in electrofluorinations at all. For example, the trifluoroacetyl group as protecting group is described in the related art only in general: in an overview of protecting groups in Journal of the Chemical Society, Perkin Transactions 1, 1999, Issue 24, pp. 1589-1615; in amino acid synthesis (for overview see Chem. Rev. 2009, 109, pp. 2455-2504); and with hazard conditions like nitration (see Chemistry Department, University of Bath in Propellants, Explosives, Pyrotechnics 32, No. 1 (2007), page 20-31, DOI: 10.1002/prep.200700004 and WO2004076384).

The trifluoroacetyl group is stable under acid conditions, but can be quite easily removed under basic conditions. In general, any perfluoro acetyl group fulfills the protecting group requirements, but due to cost and environmental reasons, the trifluoro acetyl group is the most typical one.

Perfluoro sulfonyl groups behave technically similar as protecting group, but are little less typical, as compared to the trifluoroacetyl group, mainly due to cost reasons; but cost reason becomes less important if more often is recycled and depends on recycling rate. However, during deprotecting some side reactions are possible, as free perfluoro sulfonic acids add a risk to induce rearrangement reactions and other side reactions in partial fluorinated intermediate stages and the final nonafluoro-tert-butyl alcohol (perfluoro-t-butanol; perfluoro-t-BuOH) product.

According to this disclosure, in tert-butyl alcohol (t-butanol; t-BuOH) can be easily acylated, for example, with the typical trifluoro acetyl group without any catalyst or activator by using trifluoro acetyl chloride (TFAC) or trifluoro acetic acid anhydride (TFAH), whereas the use of TFAC has the advantage that gaseous HCl leaves any reaction apparatus, and thus, a separation step to obtain the trifluoro acetylated nonafluoro-tert-butyl alcohol (perfluoro-t-butanol; perfluoro-t-BuOH) can be saved.

The synthesis of trifluoroacetic acid tert-butyl ester is well known, and using trifluoroacetic acid anhydride (TFAH) is disclosed in Tetrahedron Letters (2002), 43(48), 8687-8691. Also using trifluoroacetic acid is disclosed, e.g., in Journal of Molecular Catalysis (1986), 37(1), 45-52. The formation of trifluoroacetic acid tert-butyl ester as a side product by deprotection of CF₃—CO— protected peptides in trifluoroacetic acid (TFA) is described, e.g., in International Journal of Peptide & Protein Research (1978), 12(5), 258-68.

The use of trifluoroacetylchloride (TFAC) for the synthesis of trifluoroacetic acid tert-butyl ester, or alternatively the use of triflyl chloride, respectively, is new and has certain advantages as in both cases HCl formed in the deprotecting reaction just leaves the reaction apparatus as a gas, and leaves behind the targeted product as residue, which then can be used without any need of isolation and/or further purification. However, of course if desired the residue of the targeted product can be subjected to suitable isolation and/or purification method. The deprotection, taking place after fluorination step according to the present disclosure, is quite simple and can be performed under basic conditions. The trifluoroacetic acid and its salts, which are formed during deprotection, can be recycled as TFA which either is transferred to TFAC or TFAH according to literature procedures, e.g., to close the loop and not to waste the valuable protecting group materials. The 1,1-dimethylethyl triflate ((CH₃)₃COSO₂CF₃) is mentioned in Journal of Organic Chemistry 47 (1982), p. 4577, and the perfluoro-tert-butyl triflate, the product after fluorination, and preparation was already described by 3M in 1976 in U.S. Pat. No. 3,981,928 by reacting perfluoro-t-butanol (perfluoro-t-BuOH) with triflic anhydride. In said disclosure the perfluoro-t-butanol (perfluoro-t-BuOH) itself, i.e., the targeted product of this disclosure, was prepared according to the following reaction scheme:

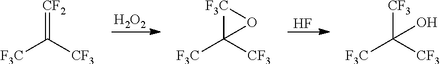

Also the perfluoro-tert-butyl trifluoroacetate was already prepared as disclosed in U.S. Pat. No. 3,981,928 out of perfluoro-t-BuOH and TFAH, and also the perfluoro-tert-butyl trifluorosulfonate was prepared out of perfluoro-t-BuOH and triflic anhydride. But as the perfluoroisobutylene as starting material is very expensive and is quite difficult to produce, e.g., out of HCFC-124 (CF₃—CFClH), a not isolated intermediate of the synthesis of the refrigerant HFC-125 (CF₃—CF₂H) by pyrolysis reaction with chlorodifluormethane in a gold-lined reactor at 800° C. (WO2002006193) this is not a suitable economic synthesis route for large scale industrial production.

The nonafluoro-tert-butyl alcohol, commonly also known as perfluoro-t-butanol (perfluoro-t-BuOH), made by the new process of the present application can be used in any application technical application as commonly known in the related art. For example, as already mentioned, it can be used to produce the peroxide of perfluoro-t-butanol which in turn can be used as a substitute for SF₆. The perfluoro-t-butanol (perfluoro-t-BuOH) can also be used in batteries. The fact is that battery applications require a very high level of purity (at least 99.9%) for the perfluoro-t-butanol (perfluoro-t-BuOH). Accordingly, here the processes of the present disclosure provide additional advantage in terms of yields and purities achieved. Although, the demands for using perfluoro-t-butanol (perfluoro-t-BuOH) as a substitute for SF₆, and in case of using it in the field of agro-chemicals or pharmaceuticals, are less than for battery application, e.g., as lower purities are sufficient as further conversions with cleaning steps follow in the manufacture of SF₆ and of agro-chemicals or pharmaceuticals, yet the processes of the present disclosure provide additional advantages by overcoming the mentioned disadvantages of the related art processes, and also in terms of yields and purities.

### Definitions

Some further definitions of terms used herein are given here below, in addition to those terms already defined in the summary of the disclosure.

A substance is “anhydrous” if it contains no water. Many processes in chemistry can be impeded by the presence of water; therefore, it is important that water-free reagents and techniques are used. In practice, however, it is very difficult to achieve perfect dryness; anhydrous substances will gradually absorb water (humidity) from the atmosphere so they must be stored carefully.

“Anhydrous hydrogen fluoride” (“AHF”) is prepared by converting the mineral fluorspar (CaF₂) typically in a rotary kiln with H₂SO₄ to HF and CaSO₄. As H₂SO₄ is a drying agent anyway, the produced anhydrous hydrogen fluoride (AHF) might have only traces of moisture and only resulting from external contamination, e.g., occurring in storage tank, in connection/disconnection of pipes, and from moisture traces resulting from inert gas(es) and/or solvent(s) potentially used in the processing and/or application of anhydrous hydrogen fluoride (AHF).

As an example, a typical specification of commercially available anhydrous hydrogen fluoride (AHF) is given herein after (inspection method GB7746-2011; China National Standards, Anhydrous Hydrogen Fluoride for Industrial Use):

| Constituent | Index | Result |
|---|---|---|
| HF-%≥ | 99.90 | 99.99 |
| H₂O%≤ | 0.005 | 0.0002 (corresponding |
| | | to 0.2 ppm) |
| H₂SiF₆%≤ | 0.005 | 0.00086 |
| SO₂%≤ | 0.002 | 0.00052 |
| H₂SO₄%≤ | 0.005 | 0.00084 |
| As (ppm)≤ | 5 | 1.0 |
| Color | colorless | colorless |

In the electrofluorination process of the present disclosure traces of water (H₂O) are tolerated of even up to about 100 ppm. Such traces of water can even contribute to an advantageous increase in the conductivity of the hydrogen fluoride (HF) used in the electrofluorination process. However, such traces of water must be counter-balanced against the disadvantage of too high traces water (H₂O) in the electrofluorination process, i.e., potential corrosion in the electrolytic cell and possibly increased consumption of the electrode material(s). Therefore, the term “water-free” or “essentially water-free,” or similar terms, in the context of the present disclosure denote a water content (traces) of at maximum about 100 ppm (≤100 ppm), typically of at maximum about 50 ppm (≤50 ppm), more typically of at maximum about 40 ppm (≤40 ppm) or 30 ppm (≤30 ppm), and even more typically of at maximum about 20 ppm (≤20 ppm).

Accordingly, the term “anhydrous hydrogen fluoride” (“AHF”) means an essentially water-free hydrogen fluoride with traces of water of at maximum about 100 ppm (≤100±5 ppm), typically of at maximum about 50 ppm (≤50±5 ppm), more typically of at maximum about 40 ppm (≤40±5 ppm) or 30 ppm (≤30±5 ppm), and even more typically of at maximum about 20 ppm (≤20±5 ppm). In particular, the term “anhydrous hydrogen fluoride” (“AHF”) “essentially water-free hydrogen fluoride” (or similar terms, e.g., “anhydrous HF,” “water-free HF” or “water-free hydrogen fluoride”) thus means “anhydrous hydrogen fluoride for industrial use,” and especially that the hydrogen fluoride (HF) typically contains at maximum approximately 20 ppm of water (20±1 ppm), typically at maximum approximately 15 ppm of water (15±1 ppm), and more typically at maximum approximately 10 ppm (10±1 ppm), of water traces.

In liquid anhydrous HF, self-ionization occurs: 3 HF [symbol] H₂F⁻+HF₂⁻ which forms an extremely acidic liquid (H₀=−15.1). The Hammett acidity function (H₀) is a measure of acidity that is used for very concentrated solutions of strong acids. Self-ionization or molecular autoionization is a reaction between molecules of the same substance to produce ions. If a pure liquid partially dissociates into ions, it is said to be self-ionizing. Normally the oxidation number on all atoms in such a reaction remains unchanged. Such autoionization can be protic (H⁺ transfer), and any solvent that contains a labile H⁺ (proton) is called a protic solvent. The molecules of such protic solvent readily donate (H⁺) to solutes, often via hydrogen bonding. Protic solvents often undergo some autoionization, and here in context of the disclosure proton transfer between two HF combines with homoassociation of F⁻ and a third HF to form HF₂⁻.

The numerical ranges disclosed herein include all values from, and including, the lower and upper value. For ranges containing explicit values (e.g., 1 to 7), any subrange between any two explicit values is included (e.g., 1 to 2; 2 to 6; 5 to 7; 3 to 7; 5 to 6; etc.).

The terms “comprising,” “including,” “having,” and their derivatives, are not intended to exclude the presence of any additional component, step or procedure, whether or not the same is specifically disclosed. In order to avoid any doubt, all compositions claimed through use of the term “comprising” may include any additional additive, adjuvant, or compound, whether polymeric or otherwise, unless stated to the contrary. In contrast, the term, “consisting essentially of” excludes from the scope of any succeeding recitation any other component, step, or procedure, excepting those that are not essential to operability. The term “consisting of” excludes any component, step, or procedure not specifically delineated or listed. The term “or,” unless stated otherwise, refers to the listed members individually as well as in any combination. Use of the singular includes use of the plural and vice versa.

The term “vol.-%” as used herein means “% by volume.” Unless otherwise stated, all percentages (%) as used herein denote “vol.-%” or “% by volume,” respectively.

### Electrofluorination:

An electrolytic cell is an electrochemical cell that uses electrical energy to drive a non-spontaneous redox reaction. The terms “electrolytic cell,” “electrolysis cell,” “electrolysis” or similar terms, respectively, are used synonymously in the context of the present disclosure. An electrolytic cell, for example, is used to decompose chemical compounds, in a process called electrolysis. Examples of electrolysis in the related art are the decomposition of water into hydrogen and oxygen, and bauxite into aluminum and other chemicals.

In the electrofluorination according to the present disclosure electrolysis is performed in the presence of hydrogen fluoride (HF), and means that hydrogen fluoride (HF) is electrolyzed to form hydrogen and fluorine, and wherein the fluorine formed in the electrolysis then is used for fluorinating a compound, i.e., in case of the present disclosure for fluorinating a hydroxyl group protected t-butanol compound.

An electrolytic cell has three component parts: an electrolyte and two electrodes, a cathode and an anode.

An electrolyte is a substance that produces an electrically conducting solution when dissolved in a polar solvent. The dissolved electrolyte separates into cations and anions, which disperse uniformly through the polar solvent. Electrically, such a solution is neutral. If an electric potential is applied to such a solution, the cations of the solution are drawn to the electrode that has an abundance of electrons, while the anions are drawn to the electrode that has a deficit of electrons. The movement of anions and cations in opposite directions within the solution amounts to a current. This includes most soluble salts, acids, and bases. A substance that dissociates into ions in solution acquires the capacity to conduct electricity. Fluoride, potassium, calcium, magnesium are some examples of electrolytes. The electrolyte in context of the present disclosure is anhydrous hydrogen fluoride (anhydrous HF, “AHF”), which can dissociate fluorine ions itself, or in which optionally further fluorine ions can be dissolved, e.g., resulting from inorganic fluorine salts.

When driven by an external voltage applied to the electrodes of the electrolytic cell, the ions in the electrolyte are attracted to an electrode with the opposite charge, where charge-transferring (also called faradaic or redox) reactions can take place. Only with an external electrical potential (i.e., voltage) of correct polarity and sufficient magnitude an electrolytic cell can decompose a normally stable or inert chemical compound. Thus, the electrical energy provided to the electrolytic cell produces a chemical reaction which would not occur spontaneously otherwise.

In an electrolytic cell, the cathode is where the negative polarity is applied to drive the cell. Common results of reduction at the cathode are hydrogen gas. An anode is an electrode through which the conventional current enters into a polarized electrical device. This contrasts with a cathode, an electrode through which conventional current leaves an electrical device. In an electrolytic cell, the anode is the wire or plate having excess positive charge. Consequently, anions will tend to move towards the anode where they can undergo oxidation.

As stated before, the term “fluorination in hydrogen fluoride,” “fluorination in HF,” “electrolysis in hydrogen fluoride” or “electrolysis in HF” (e.g., “HF/electrolysis” or similar terms) means or is used, respectively, synonymously with the term “electrofluorination” or “electrochemical fluorination,” respectively. Electrofluorination or electrolysis, respectively, in hydrogen fluoride (HF) can take place in hydrogen fluoride (HF) solution or suspension.

As stated before, the term “HF” or “hydrogen fluoride,” respectively, is meant to denote always “anhydrous hydrogen fluoride” (“AHF”), unless expressively stated otherwise.

HF (hydrogen fluoride) serves as solvent, also as electrolyte, and also serves as fluorinating agent (source of fluorine atoms) in the electrofluorination (ECF). HF (hydrogen fluoride) may serve as the sole fluorinating agent (source of fluorine atoms) in the electrofluorination (ECF).

Typically, in this disclosure, and, e.g., as described in the experimental trials given herein (e.g., see Examples), conductivity of the electrolyte, e.g., of HF (hydrogen fluoride), can be increased by adding metal halogenide(s), typically metal fluoride(s), or any combination thereof, but as stated before the electrofluorination (ECF) also can be performed without addition of such metal halogenide(s) or typical metal fluoride(s), respectively.

As a representative example, but without limiting to this even typical example, the metal halogenide(s), typically the metal fluoride(s), is potassium fluoride (KF) and is added into the HF (hydrogen fluoride). Although, the electrofluorination (ECF) can be performed without addition of such metal halogenide(s) or typical metal fluoride(s), respectively, in general addition of potassium fluoride (KF) or any other fluoride salt seems to give some better results (not further optimized) than without such addition. Also amine×3 HF (e.g., NEt₃×3 HF; NEt₃ means tri-ethanol amine) can be used as hydrogen fluoride (HF) source and electrolyte, but also this gives worse results vs. addition of potassium fluoride (KF) to anhydrous HF.

Typically, a suitable inorganic fluorine salt or combination of inorganic fluorine salts is added as additional fluorinating agent, and for increasing the conductivity. For example, the inorganic fluorine salt is selected from the group consisting of alkaline metal fluorides, alkaline earth metal fluorides, and ammonium fluoride (NH₄F). Suitable alkaline metal fluorides are lithium (Li) fluoride, sodium (Na) fluoride, and potassium (K) fluoride. Suitable alkaline earth metal fluorides are beryllium (Be) fluoride, magnesium (Mg) fluoride, calcium (Ca) fluoride, strontium (Sr) fluoride, and barium (Ba) fluoride. Ammonium fluoride (NH₄F) may also be considered and used as inorganic fluorine salt, but alkali fluorides and/or earth alkali fluorides are typical over ammonium fluoride (NH₄F). Typically, the inorganic fluorine salts in the process of the present disclosure are used in its spray dried form.

The electrofluorination according to the present disclosure can be performed in any electrolysis cell known to the person skilled in the field, which is adapted to be resistant against hydrogen fluoride (HF).

In a particular aspect of the present disclosure the electrofluorination is performed in a so called Simmons electrolysis cell, for example, as summarized in Houben Weyl 4^{th} Edition Vol V/3 (1962), Fluorine and Chlorine Compounds, by authors from Hoechst Farbwerke Frankfurt, page 38 ff., and as originally developed by 3M and as disclosed in U.S. Pat. No. 2,519,983 with an iron/steel cathode.

In the electrofluorination process hydrogen is generated at the cathode, and where at a Ni-anode the fluorination takes place. A current is applied (5-6 V, 0.02 A/cm²), which is slightly below the needed current for producing elemental fluorine gas, and at a temperature range of 0° C. to 20° C. It is emphasized that a diaphragm is not needed in the electrofluorination of the present disclosure.

The applied electrofluorination process of the disclosure and as described herein is according to the disclosure by Hollitzer and Sartori, in Chem. Ing. Tech. 58 (1986) page 31-38, and which disclosure here is incorporated by reference in its entirety; and it is applied to flow electrolysis as in general used for fluorine free compounds, and as disclosed in Chem. Rev. 2018, 118, 9, 4573-4591 (doi.org/10.1021/acs.chemrev.7b00360; Pletcher et al., Open Access Article on “Flow Electrolysis Cells for the Synthetic Organic Chemistry Laboratory”), and which disclosure here is incorporated by reference in its entirety, and is applied to electrofluorination (ECF) of the present disclosure.

In context of the present disclosure, the term “flow,” or “flow electrolysis” or similar terms, respectively, means performing electrolysis under flow chemistry conditions.

Flow chemistry is also known as continuous flow or plug flow chemistry. It involves a chemical reaction run in a continuous flow stream. The process offers potential for the efficient manufacture of chemical products. Reactants, optionally, are first pumped into a mixing device, and then flow continues through a temperature controlled reactor until the reaction is complete. The reactor can be a simple pipe/tube, e.g., a coil reactor, or a complex micro-structured device. The (optional) mixing device and reactor are maintained at the precise temperature to promote the desired reaction. The reactants here in context of the present disclosure are also be exposed to an electrical flux (electrical conductivity) to promote an electrochemical reaction, i.e., to promote the electrochemical fluorination reaction or electrofluorination (ECF), respectively, of the present disclosure.

However, “flow chemistry” or “flow electrolysis,” respectively, hitherto was not known in context of electrofluorination, and such “flow chemistry” or “flow electrolysis,” respectively, hitherto was never applied for electrofluorination. Accordingly, applying electrofluorination (ECF), in particular applying electrofluorination (ECF) in “flow,” as in the context of the present disclosure in general, and as especially in combination with the hydroxyl group protecting and deprotecting chemistry is novel, and provides for a surprisingly advantageous process for manufacturing the targeted compounds of the present disclosure.

The design of such a flow electrochemical reactor(s) for electrofluorination is made and by and commercially available from Vapourtec (www.vapourtec.com/flow-chemistry; see also Example 4 herein below).

In particular, in context of the present disclosure, the term “flow,” or “flow electrolysis” or similar terms, respectively, means either (i) continuous circulation of electrolyte (e.g., with no feed of feeding fresh starting material) in an electrolysis reactor or (ii) continuous one time pass-by of starting material to be electrofluorinated at an anode/cathode mix of electrodes in an electrolysis reactor. Continuous circulation of electrolyte according to (i) in an electrolysis reactor has the advantage that the circulation of electrolyte removes residues from electrode(s) and at least reduces or even avoids over-fluorination and/or decomposition (note: C—C bonds break when residence time at electrode(s) is to too long). Continuous one time pass-by according to (ii) of starting material to be electrofluorinated at an anode/cathode mix of electrodes in an electrolysis reactor is typical.

Continuous one time pass-by according to (ii) of starting material to be electrofluorinated at an anode/cathode mix of electrodes in an electrolysis reactor is even more preferable, when one time pass-by of starting material is performed in an electrolysis reactor having a kind of sandwich microreactor comprising an anode/cathode mix and a microchannel design, e.g., a microreactor design. Such sandwich microreactor meanwhile is quite common in the related art for performing thermal reactions, and the design and working principle thereof well known to the person skilled in the art, but hitherto was never applied for electrofluorination.

Each of the (i) continuous circulation of electrolyte (e.g., with no feed of feeding fresh starting material) in an electrolysis reactor or (ii) continuous one time pass-by of starting material to be electrofluorinated at an anode/cathode mix of electrodes in an electrolysis reactor, was hitherto not known, and hitherto was never applied for electrofluorination.

Also, electrofluorination in such sandwich microreactor, as described here above, hitherto was not known, and such sandwich microreactor hitherto was never applied for electrofluorination. In a most typical aspect of the disclosure the electrofluorination process is performed with a floating electrolyte, e.g., as to some minor extent described in early days by Phillips Petroleum in U.S. Pat. No. 4,146,443. However, the electrode surface, which was only at the walls of the apparatus described by Phillips Petroleum, was much too small to achieve industrially useful performances.

The electrofluorination (ECF) process where volatile fluorinated products are taken out of the gas phase of the electrolysis cell is called CAVE-Phillips process (CAVE (Carbon Anode Vapor phase Electrochemical fluorination) and is disclosed in Organofluorine Chemistry, 1994, pp. 121-143.

The electrofluorination (ECF) of trifluoroacetic acid isopropyl ester was already disclosed by Phillips Petroleum in U.S. Pat. No. 3,900,372. However, this electrofluorination of trifluoroacetic acid isopropyl ester was performed in an industrially not useful electrofluorination (ECF) cell, and was not performed as a flow process.

In an aspect of the disclosure a Ni-anode (nickel-anode) is also possible in the electrofluorination (ECF) process, and is even typical if the Ni-anode (nickel-anode) is made out of porous nickel (Ni) material.

As electrochemistry is named “green chemistry” several study investigations are going on, where Fraunhofer IMM, Germany, is among the leaders (see IMM-Production Of Chemicals By Electrochemical Microreactors.pdf (fraunhofer.de). In general, Fraunhofer IMM only suggests that their electrochemical procedures are broadly applicable in fields ranging from water treatment, hydrogen peroxide production to a multitude of electroorganic synthesis like the oxidation of alcohols, phenols, aldehydes, halogenation of aromatics, alkoxylation reactions, the synthesis of aromatic aldehydes, C—C cross coupling reactions and to reduction of nitro groups to name of few. Another application mentioned by Fraunhofer IM is the cation pool and cation flow method as modern organic synthesis approach. For example, Fraunhofer IMM here also reports about experiences gained in the field of the Kolbe electrolysis in microreactors, which Kolbe electrolysis is formally a decarboxylative dimerization of two carboxylic acids or carboxylate ions.

However, such electrochemical microreactor equipment hitherto is unknown for applications in electrofluorination (ECF), and in a particular aspect of the present disclosure surprisingly it was found that such equipment as electrochemical microreactor advantageously can be also used in electrofluorination (ECF), which use in electrofluorination (ECF) is novel.

Accordingly, in this aspect, the present disclosure also pertains to a process comprising an electrofluorination (ECF) process step, wherein a hydroxyl group protected tert-butyl alcohol compound (hydroxyl group protected t-butanol or t-BuOR, respectively, wherein R is a hydroxyl group protecting group as defined herein) is subjected to a fluorination step under electrofluorination conditions in an electrochemical microreactor, in particular as described herein. Further, the present disclosure also pertains to a process wherein the targeted nonafluoro-tert-butyl alcohol, commonly also known as perfluoro-t-butanol (perfluoro-t-BuOH), is manufactured by a process comprising the steps of protecting the hydroxyl-function (OH-function) in t-butanol followed by a deprotecting step of the hydroxyl-function (OH-function) after having performed a fluorination step under electrofluorination conditions in an electrochemical microreactor, in particular as described herein.

### Electrochemical Microreactor:

An electrochemical microreactor (cf. those described by Fraunhofer IMM) used in one aspect of the present disclosure typically is characterized by small electrode distances and great surface-to-volume-ratios as key features. Such a so-called thin-gap electrochemical microreactor provides the constructive features and related advantages: This electrochemical microreactor is designed as plate stack reactor as fundament for a flexible reactor concept. The plates bear the structured electrodes typically on both sides and are equipped with an integrated heat exchanger. A combination of additive manufacturing to realize the base plates with their complex fluid structures, surface coating (e.g., with PTFE) of the plates for electric insulation, milling to create the micro channels on the plates surface and electroplating to deposit different electrode materials are used to realize the versatile electrochemical microreactor. The basic reactor plates are characterized, for example, as follows: electrode outer dimension of 100 mm×118 mm; active electrode surface per structured plate side of 53.6 cm² provided via 67 microchannels; channel dimensions of 800 μm in width, 100 μm in depth and 100 mm in length; channel volume per structured plate side amounts to 0.5 cm³. The reactor is designed for operation conditions up to 200° C. and up to 100 bar, for electrolyte flow rates up to 100 ml min⁻¹, and for coolant flow rates up to 50 ml min⁻ per electrode assembly. The reactor concept allows a multitude of operation possibilities, for example: use of 1 to 9 electrode assembly units in the overall reactor housing; operation of the electrode assemblies as mono or bipolar cells; operation of the electrode assemblies as undivided or divided cell (e.g., by use of diaphragms or ion exchange membranes; e.g., PEM); parallel, serial or mixed operation of the electrode assemblies (therewith, numbering-up and scale-up possibilities are also given); individual designation of electrode assemblies is feasible when using a multichannel galvanostat.

As electrode materials and/or electrode coatings stainless steel, platinum and boron doped diamond (BDD) can be in place. Materials as, e.g., nickel, graphite, glassy carbon, lead or lead oxide are in principle also described by Fraunhofer IMM as feasible as electrode materials. In context of the present disclosure nickel is a typical electrode material and/or electrode coating. More typically, in the context of the present disclosure an electrochemical microreactor is used, wherein the anode(s) is Ni-anode (nickel-anode), typically wherein the Ni-anode(s) (nickel-anode) is made out of porous nickel (Ni) material.

The electrochemical microreactor is further described in WO 00/15872 A1 (Fraunhofer), and reaction conditions applied in such electrochemical microreactor is further described in WO 2016/170075 A1 (Fraunhofer), as exemplified for a Kolbe reaction.

### Aspects of the Disclosure

In one aspect the present disclosure pertains to a hydroxyl group protected and methyl group perfluorinated tert-butyl alcohol ester compound of formula (II),

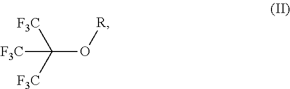

wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue;
and wherein the hydroxyl group protected and methyl group perfluorinated tert-butyl alcohol ester compound of formula (II) is obtained by an electrofluorination (ECF) process out of a hydroxyl group protected tert-butyl alcohol compound of formula (III),

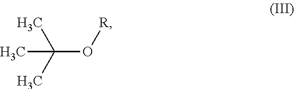

wherein the substituent R in compound of formula (III) has the same meaning
as the substituent R in compound of formula (II).

In another aspect the present disclosure pertains to the use of a hydroxyl group protected tert-butyl alcohol ester compound of formula (II),

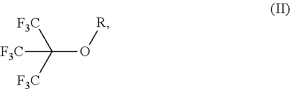

wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue;
and wherein the hydroxyl group protected and methyl group perfluorinated tert-butyl alcohol ester compound of formula (II) is obtained by an electrofluorination (ECF) process out of a hydroxyl group protected tert-butyl alcohol compound of formula (III),

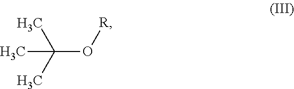

wherein the substituent R in compound of formula (III) has the same
meaning as the substituent R in compound of formula (II);
in the manufacture of a nonafluoro-tert-butyl alcohol compound of formula (I),

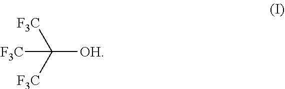 [SMILES: OC(C(F)(F)F)(C(F)(F)F)C(F)(F)F]

In a further aspect the present disclosure pertains to a process for the manufacture of a hydroxyl group protected nonafluoro-tert-butyl alcohol ester compound of formula (II),

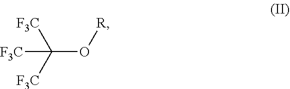

wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue,
comprising or consisting of the steps of:
(i) an electrofluorination (ECF) step (B) of reacting the hydroxyl group protected tert-butyl alcohol compound of formula (III),

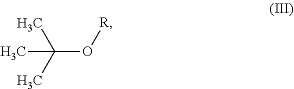

wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue,
in the presence of anhydrous hydrogen fluoride (AHF) to obtain a hydroxyl group protected nonafluoro-tert-butyl alcohol ester compound of formula (II);
(ii) optionally, in parallel and/or subsequent to the electrofluorination (ECF) step (B), an isolating and/or purifying step (D) to obtain the isolated and/or purified hydroxyl group protected nonafluoro-tert-butyl alcohol ester compound of formula (II).

The process according to the disclosure, as defined herein, also pertains to the manufacture of a hydroxyl group protected nonafluoro-tert-butyl alcohol ester compound of formula (II), wherein the hydroxyl group protected tert-butyl alcohol compound of formula (III) prior to its use in the electrofluorination (ECF) step (B) is prepared by a process comprising or consisting of the steps of a protecting reaction step (A) of reacting tert-butyl alcohol compound of formula (IV),

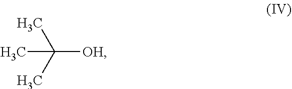

with a hydroxyl group protecting agent of formula R—X (V), wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue, and
X denotes a hydrogen atom, a halogen atom or an —O—R group, to obtain a hydroxyl group protected tert-butyl alcohol compound of formula (III) as defined herein above,
and the hydroxyl group protected tert-butyl alcohol compound of formula (III) obtained in step (A), with or without isolating and/or purifying, is subjected to the electrofluorination (ECF) step (B) as defined herein above.

In still a further aspect the present disclosure pertains to a process for the manufacture of a nonafluoro-tert-butyl alcohol compound of formula (I),

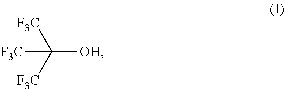 [SMILES: OC(C(F)(F)F)(C(F)(F)F)C(F)(F)F]

via a hydroxyl group protected nonafluoro-tert-butyl alcohol ester compound of formula (II) as defined herein above, comprising or consisting of the steps of:
(i) a protecting reaction step (A) of reacting tert-butyl alcohol compound of formula (IV),

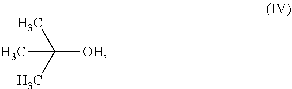

with a hydroxyl group protecting agent of formula R—X (V), wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue, and
X denotes a hydrogen atom, a halogen atom or an —O—R group, to obtain a hydroxyl group protected tert-butyl alcohol compound of formula (III),

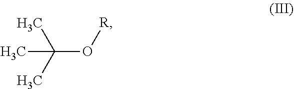

wherein the substituent R in formula (III) has the same meaning as indicated
here before for the substituent R in formula (V);
and with or without isolating and/or purifying hydroxyl group protected tert-butyl alcohol compound of formula (III),
(ii) electrofluorination (ECF) step (B) of reacting the hydroxyl group protected tert-butyl alcohol compound of formula (III) obtained in step (A) in the presence of anhydrous hydrogen fluoride (AHF) to obtain a hydroxyl group protected nonafluoro-tert-butyl alcohol ester compound of formula (II),

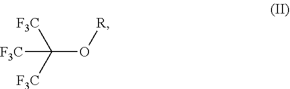

wherein the substituent R in formula (II) has the same meaning as indicated here before for the substituent R in formula (III);
and with or without isolating and/or purifying the hydroxyl group protected nonafluoro-tert-butyl alcohol ester compound of formula (II),
(iii) a deprotecting reaction step (C) of removing the hydroxyl group protecting group R from the hydroxyl group protected nonafluoro-tert-butyl alcohol ester compound of formula (II) obtained in step (B) to obtain the nonafluoro-tert-butyl alcohol compound of formula (I);
(iv) optionally, in parallel and/or subsequent to the deprotecting reaction step (C), an isolating and/or purifying step (D) to obtain the isolated and/or purified nonafluoro-tert-butyl alcohol compound of formula (I).

In still another aspect the present disclosure pertains to a process for the manufacture, as defined herein, of a nonafluoro-tert-butyl alcohol compound of formula (I),

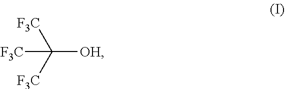 [SMILES: OC(C(F)(F)F)(C(F)(F)F)C(F)(F)F]

via a hydroxyl group protected tert-butyl alcohol ester compound of formula (II) as defined herein above, comprising or consisting of the steps of:
(i) electrofluorination (ECF) step (B) of reacting a hydroxyl group protected tert-butyl alcohol compound of formula (III),

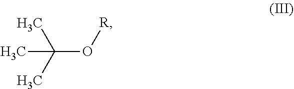

wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue,
in the presence of anhydrous hydrogen fluoride (AHF) to obtain a hydroxyl group protected tert-butyl alcohol ester compound of formula (II),

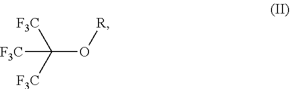

wherein the substituent R in formula (II) has the same meaning as indicated here before for the substituent R in formula (III),
and with or without isolating and/or purifying the hydroxyl group protected nonafluoro-tert-butyl alcohol ester compound of formula (II),
(ii) a deprotecting reaction step (C) of removing the hydroxyl group protecting group R from the hydroxyl group protected nonafluoro-tert-butyl alcohol ester compound of formula (II) obtained in step (B) to obtain the nonafluoro-tert-butyl alcohol compound of formula (I);
(iii) optionally, in parallel and/or subsequent to the deprotecting reaction step (C), an isolating and/or purifying step (D) to obtain the isolated and/or purified nonafluoro-tert-butyl alcohol compound of formula (I).

In yet another aspect the present disclosure pertains to a process for the manufacture of a nonafluoro-tert-butyl alcohol compound of formula (I),

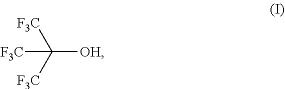 [SMILES: OC(C(F)(F)F)(C(F)(F)F)C(F)(F)F]

via a hydroxyl group protected nonafluoro-tert-butyl alcohol ester compound of formula (II) as defined herein above, comprising or consisting of the steps of:
(i) a deprotecting reaction step (C) of removing the hydroxyl group protecting group R from the hydroxyl group protected nonafluoro-tert-butyl alcohol ester compound of formula (II)

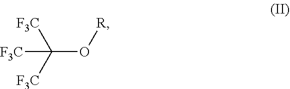

wherein the substituent R in formula (II) has the same meaning as indicated here before for the substituent R in formula (III),
to obtain the methyl group perfluorinated nonafluoro-tert-butyl alcohol compound of formula (I);
(ii) optionally, in parallel and/or subsequent to the deprotecting reaction step (C), an isolating and/or purifying step (D) to obtain the isolated and/or purified nonafluoro-tert-butyl alcohol compound of formula (I).

In still a further aspect the present disclosure pertains to a process for the manufacture of a hydroxyl group protected nonafluoro-tert-butyl alcohol ester compound of formula (II),

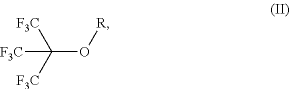

wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue,
comprising or consisting of the steps of:
(i) a protecting reaction step (A) of reacting tert-butyl alcohol compound of formula (IV),

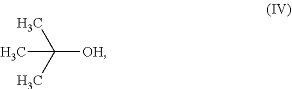

with a hydroxyl group protecting agent of formula R—X (V), wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue, and
X denotes a hydrogen atom, a halogen atom or an —O—R group, to obtain a hydroxyl group protected tert-butyl alcohol compound of formula (III),

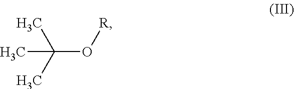

wherein the substituent R in formula (III) has the same meaning as indicated here before for the substituent R in formula (V);
and with or without isolating and/or purifying hydroxyl group protected tert-butyl alcohol compound of formula (III),
(ii) electrofluorination (ECF) step (B) of reacting the hydroxyl group protected tert-butyl alcohol compound of formula (III) obtained in step (A) in the presence of anhydrous hydrogen fluoride (AHF) to obtain a hydroxyl group protected nonafluoro-tert-butyl alcohol ester compound of formula (II);
(iv) optionally, in parallel and/or subsequent to the electrofluorination (ECF) step (B), an isolating and/or purifying step (D) to obtain the isolated and/or purified hydroxyl group protected nonafluoro-tert-butyl alcohol ester compound of formula (II).

In yet a further aspect the present disclosure pertains to a process for the manufacture of a hydroxyl group protected nonafluoro-tert-butyl alcohol ester compound of formula (II),

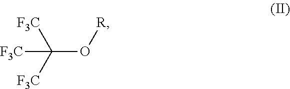

wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue,
comprising or consisting of the steps of:
(i) electrofluorination (ECF) step (B) of reacting the hydroxyl group protected tert-butyl alcohol compound of formula (III),

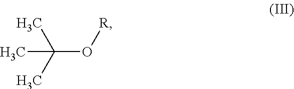

wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue,
in the presence of anhydrous hydrogen fluoride (AHF) to obtain a hydroxyl group protected nonafluoro-tert-butyl alcohol ester compound of formula (II);
(ii) optionally, in parallel and/or subsequent to the electrofluorination (ECF) step (B), an isolating and/or purifying step (D) to obtain the isolated and/or purified hydroxyl group protected nonafluoro-tert-butyl alcohol ester compound of formula (II).

The present disclosure also pertains to the use of the hydroxyl group protected and methyl group perfluorinated nonafluoro-tert-butyl alcohol ester compound of formula (II) according to the disclosure, or the process for the manufacture of a hydroxyl group protected nonafluoro-tert-butyl alcohol ester compound of formula (II) according to the disclosure, or the process for the manufacture of a nonafluoro-tert-butyl alcohol compound of formula (I) according to the disclosure, independently, R denotes a substituent selected from the group consisting of CF₃CO—, CF₂HCOCF₂ClCO—, and CF₃SO₂—.

Furthermore, the present disclosure pertains to a process for the manufacture of a hydroxyl group protected nonafluoro-tert-butyl alcohol ester compound of formula (II) according to the disclosure, or the process for the manufacture of a nonafluoro-tert-butyl alcohol compound of formula (I) according to the disclosure, independently, in the protecting reagent of formula R—X (V), independently, the group X denotes a hydrogen atom, a halogen atom or an —O—R group; typically, wherein in the protecting reagent of formula R—X (V), independently, the group X denotes a hydrogen atom, a chlorine atom or an —O—R group.

In particular aspect the present disclosure pertains to a process, wherein the electrofluorination (ECF) step is carried out in the presence of anhydrous hydrogen fluoride (AHF), and in the further presence of an inorganic halogenide salt, typically a fluoride salt, more typically in the presence of a a spray dried inorganic halogenide salt, typically spray dried inorganic fluoride salt, even more typically wherein the spray dried inorganic fluoride salt is spray dried potassium fluoride (spray dried KF). The term “halogenide” typically means chloride or fluoride; typically the “halogenide” is fluoride.

Typical is process according to the present disclosure, wherein the electrofluorination (ECF) step is carried out in the presence of anhydrous hydrogen fluoride (AHF) which is prepared by converting the mineral fluorspar (CaF₂) with H₂SO₄ to a mixture essentially comprised of hydrogen fluoride (HF) and CaSO₄, and separating off the anhydrous hydrogen fluoride (AHF) from said mixture, and which optionally further purified.

In another particular aspect the present disclosure pertains to a process according to the disclosure, wherein in the electrofluorination (ECF) step the electrolysis is performed under flow chemistry conditions, in particular as flow electrolysis. The process according to this particular aspect of the present disclosure is typical, wherein the low electrolysis is performed either (i) as continuous circulation of electrolyte (e.g., with no feed of feeding fresh starting material) in an electrolysis reactor, or (ii) as continuous one time pass-by of starting material to be electrofluorinated at an anode/cathode mix of electrodes in an electrolysis reactor.

The process according to the present disclosure is typical, wherein the electrofluorination (ECF) step is carried out in an electrochemical microreactor, typically in an electrochemical microreactor. The process according to this aspect of the present disclosure is typical, wherein the electrochemical microreactor is sandwich microreactor comprising an anode/cathode mix and a microchannel design, and wherein the continuous one time pass-by according to (ii) of starting material to be electrofluorinated at an anode/cathode mix of electrodes in an electrolysis reactor is performed.

In another aspect of the present disclosure the process as defined herein above is typical, wherein the electrofluorination (ECF) step is carried out in an electrochemical microreactor, wherein the anode(s) is Ni-anode (nickel-anode), typically wherein the Ni-anode(s) (nickel-anode) is made out of porous nickel (Ni) material.

In still another particular aspect of the present disclosure a process as defined herein above is typical, wherein the electrofluorination (ECF) step is carried out in an electrolysis cell, typically in a Simmons electrolysis cell. The process according to this aspect of the present disclosure is typical, wherein the electrofluorination (ECF) step is carried out in an electrolysis cell, typically in a Simmons electrolysis cell, wherein the anode(s) is a Ni-anode (nickel-anode), typically wherein the Ni-anode(s) (nickel-anode) is made out of porous nickel (Ni) material.

Furthermore, in a particular aspect of the present disclosure also pertains to the use of an electrochemical microreactor in an electrofluorination (ECF) step:
(i) in the manufacture of a nonafluoro-tert-butyl alcohol compound of formula (I),

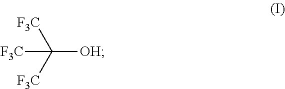 [SMILES: OC(C(F)(F)F)(C(F)(F)F)C(F)(F)F]

wherein the nonafluoro-tert-butyl alcohol compound of formula (I) is obtained by deprotecting a hydroxyl group protected tert-butyl alcohol ester compound of formula (II), as defined herein after in (ii);
or
(ii) in the manufacture of a hydroxyl group protected nonafluoro-tert-butyl alcohol ester compound of formula (II),

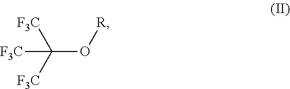

wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CH₃CO—, CF₃SO₂—, CF₂ClSO₂—, CFCl₂SO₂—, CCl₃SO₂—, CF₂HSO₂—, CFH₂SO₂—, CH₃SO₂—, PfCO— and PfSO₂—, and wherein Pf denotes a partially or perfluorinated C2-C4 residue;
and wherein the hydroxyl group protected and methyl group perfluorinated tert-butyl alcohol ester compound of formula (II) as defined under (ii) is obtained by electrofluorination (ECF) process,
   typically by electrofluorination (ECF) process in the presence of anhydrous hydrogen fluoride (AHF), and more typically by electrofluorination (ECF) process in the presence of anhydrous hydrogen fluoride (AHF) and in the further presence of an inorganic halogenide salt, typically a fluoride salt, more typically in the presence of a spray dried inorganic halogenide salt, typically a spray dried inorganic fluoride salt, and even more typically wherein the spray dried inorganic fluoride salt is spray dried potassium fluoride (spray dried KF),
out of a hydroxyl group protected tert-butyl alcohol compound of formula (III),

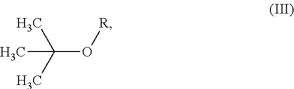

wherein the substituent R in compound of formula (III) has the same meaning as the substituent R in compound of formula (II) above.

In this aspect of the present disclosure of use of an electrochemical microreactor in an electrofluorination (ECF) step as defined herein above is typical, wherein in the electrofluorination (ECF) step an electrochemical microreactor is used, wherein the anode(s) is Ni-anode (nickel-anode), typically wherein the Ni-anode(s) (nickel-anode) is made out of porous nickel (Ni) material.

The following examples are intended to further illustrate the disclosure without limiting its scope.

### EXAMPLES

### Example 1

### Tfac (Trifluoroacetylchloride) as Protecting Group Raw Material in a Coil Reactor (Continuous Process).

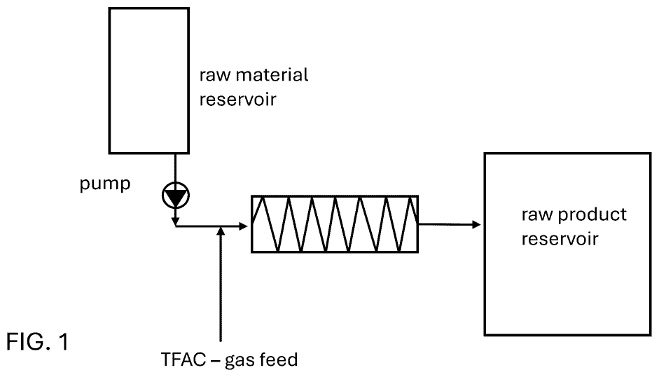

See FIG. 1 for scheme of apparatus and process.

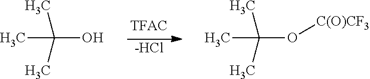

Apparatus: A coil reactor made out of Hastelloy C4 (1 m length, diameter: 0.5 cm), raw material reservoir (11), TFAC feed out of a gas cylinder, raw material reservoir (21, 145.71 stainless steel). Both feeds were equipped with Bronkhorst mass flow controllers; raw product reservoir was equipped with a pressure valve going into a basic scrubber.

Raw materials: Trifluoroacetylchloride (supplier Sinochem Lantian) and t-BuOH (technical grade, supplier Sigma Aldrich Taufkirchen).

In the coil reactor apparatus above, 96.4 g (1.3 mol) t-BuOH from Aldrich were placed into the raw material reservoir and the dosage pump started with a feed of 100 g/h. Right after start of the pump, TFAC (185.5 g/h; 1.4 mol/h) was fed as gas out of a cylinder into the coil reactor (kept with a water bath at 30° C., pressure valve set to 3 bar abs.). An exothermic reaction could be observed immediately, the material after the coil reactor was collected in the raw material reservoir kept at 0° C. by external cooling. After 58 minutes, all t-BUOH was consumed, all the feeds were stopped. Most of the formed HCl had left already into the scrubber, and the pressure of raw material reservoir was released to atmospheric pressure. A sample taken for HPLC-MS analysis indicated a quantitative conversion of t-BuOH to trifluoroacetic acid tert-butyl ester, as confirmed by GC-MS.

A sample trifluoroacetic acid tert-butyl ester was purified by distillation in a 20 cm Vigreux column at 1 bar abs. (transition temperature of 83.4° C.) to yield trifluoroacetic acid tert-butyl ester with a purity of 99.9%.

### Example 2a

### Tfac (Trifluoroacetylchloride) as Protecting Group Raw Material in a Counter-Current System (Batch Process).

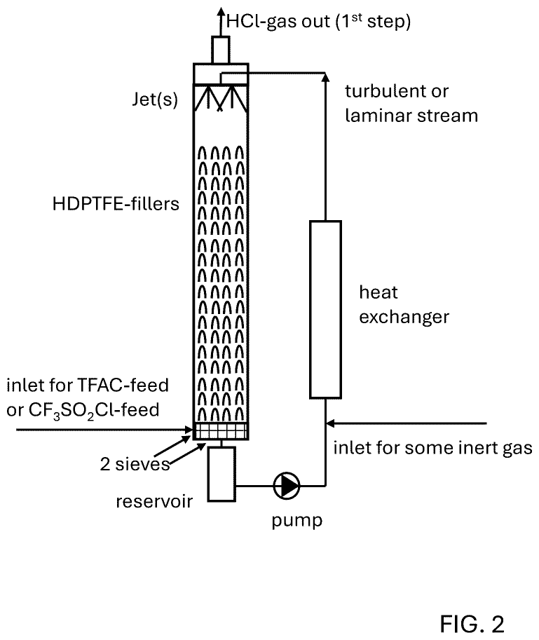

See FIG. 2 for scheme of apparatus and process.

In a batch counter-current apparatus out of Hastellloy C4 steel having a pressure valve at the top which is set to 3 bar abs. and with a liquid reservoir volume of 5 l (see drawing), a column is set on the reservoir (length 50 cm, diameter 100 mm, filled with HDPTFE fillings with diameter 10 mm) 3.01 (2.3 kg, 31.0 mol) of t-BUOH (technical grade from Aldrich) was filled in and the pump was started with a performance of 500 l/h. For the heat exchanger, a water cooling system with a water temperature of 8° C. was used. When the temperature of the t-BuOH reached 10° C. (coming from 26° C. after start of the loop), gaseous TFAC out of a cylinder was fed with a feed rate of 1000 g/h; 7.55 mol/h over a Bronkhorst mass flow controller into the gas inlet installed between raw material reservoir and column as drawn above, same inlet as used for the F₂-feed later on. After feed of 4.2 kg (32.0 mol) the TFAC the feed was stopped, 20 l N₂-inert gas was inserted at the inert gas inlet as drawn to draw out some HCl-gas residues, then the pump also was stopped after further 10 min, the pressure was released to atmospheric pressure and the content in the reservoir was analyzed by GC which showed a quantitative conversion of t-BuOH and quantitative presence of trifluoroacetic acid tert-butyl ester.

The trifluoroacetic acid tert-butyl ester was purified by fine distillation in a 20 cm Vigreux column at 1 bar abs. (transition temperature of 83.3° C.) to obtain trifluoroacetic acid tert-butyl ester in a yield of 97% and with a purity of 99.9%.

### Example 2b

TFAC (Trifluoroacetylchloride) as Protecting Group Raw Material in a Counter-Current System (Batch Process), and Counter-Current Column is Directly Placed onto the Reservoir.

The reaction is carried out like described here before in Example 2a, in a batch counter-current apparatus as described, except for the variation that the counter-current column is directly placed onto the reservoir.

In the case of a counter-current system (batch process), it is typical if the counter-current column rests directly (i.e., without diameter constriction) on the reservoir. In this case the column does not flood so easily, as compared to the process of Example 2a performed in a counter-current system (batch process) wherein the counter-current column is connected to the reservoir via a pipeline. Due to diameter of the pipeline (i.e., the narrowing of the flow diameter) the flow rate may be restricted in order to avoid undesired flooding of the counter-current column.

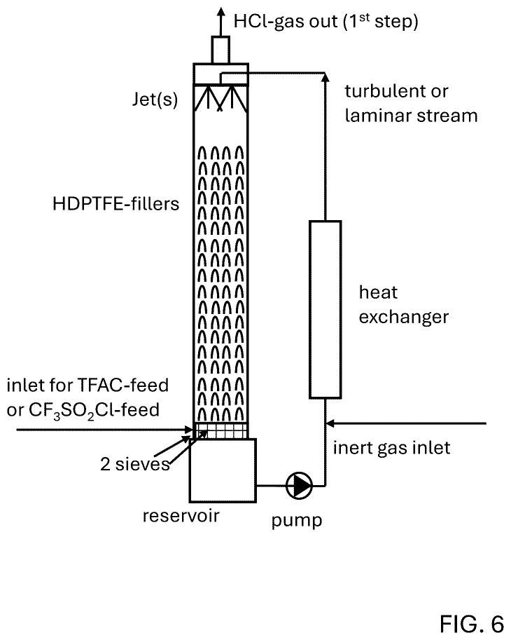

See FIG. 6 for scheme of apparatus and process with counter-current system (batch process), and counter-current column is directly placed onto the reservoir.

### Example 3

### Electrofluorination (ECF) of Trifluoroacetic Acid Tert-Butyl Ester (Batch Process) to Yield Perfluoro-Tert-Butyl Trifluoroacetate.

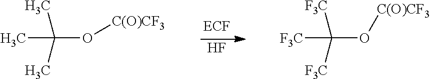

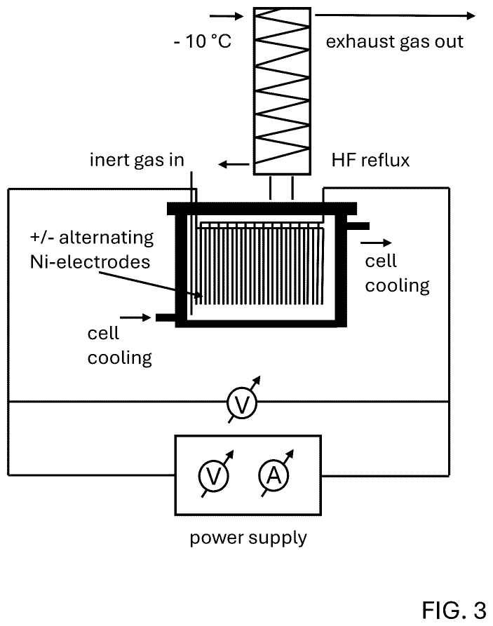

See FIG. 3 for scheme of the ECF-apparatus and electrofluorination process (batch ECF-process).

The trifluoroacetic acid tert-butyl ester obtained in Example 1 was used in an electrofluorination (ECF). A Simmons electrolysis cell was used; the working principle is described and summarized, for example, in Houben Weyl 4^{th} Edition Vol V/3 (1962), Fluorine and Chlorine compounds, by Hoechst Farbwerke Frankfurt, page 38 ff., and it was originally discovered by 3M (see, e.g., U.S. Pat. No. 2,519,983). Alternating nickel anode and cathode plates are immersed in anhydrous hydrogen fluoride (AHF) as fluorinating agent and solvent.

The Simmons electrolysis cell used in this Example consisted out of 26 Ni-plates having a width of 45 cm, a length of 30 cm. The distance between each of adjacent electrode plates was 0.5 cm, and the distance to the outer walls of the electrolysis cell was 1.0 cm. The total volume of the electrolysis cell was 18 l.

At the bottom of the electrolysis cell there were two inlets for filling and/or emptying purposes. The bottom of the cell was additionally covered by a HDPTFE plate for avoiding corrosion.

The current density was set around 4.5 A/0.093 m². The cell voltage applied was 5.25 volts.

During electrolysis, the cell temperature was kept at 3° C. by means of a double wall jacket made out of steel and comprised by the electrolysis cell.

The Simmons electrolysis cell was also equipped with a reflux condenser made out of Hastelloy, and the cooling liquid for the reflux condenser was kept at −10° C. by means of a Linde cryostat. During the electrolysis procedure (electrofluorination), a soft argon (Ar) stream was fed over the liquid phase surface to get rid of some volatile amounts of reaction products formed during electrofluorination.

The Simmons electrolysis cell below was pre-loaded with 15 l anhydrous hydrogen fluoride (AHF) as fluorinating solvent and fluorinating agent and 500 g potassium fluoride (KF) as fluorinating agent and conducting salt (spray dried KF was used) followed by 4.0 kg (23.5 mol) trifluoroacetic acid tert-butyl ester.

After duration of 5 h of electrolysis in the Simmons electrolysis cell under the conditions described above, a slight reflux could be observed.

After duration of 62 h of electrolysis (electrofluorination), the electrolysis cell was warmed up to a temperature of 30° C. and the cooling by the reflux cooler on top of the electrolysis was stopped in order to let hydrogen fluoride (HF) escape over the reflux cooler cell into a cylinder used as cooling trap (not shown).

The dark to black colored liquid residue remaining in the electrolysis cell was quenched into ice water, the organic phase separated off and after drying over Na₂SO₄ it was distilled.

The perfluoro-tert-butyl trifluoroacetate product formed in this Example was distilled off using a 20 cm Vigreux column at 1 bar abs. (transition temperature of 93° C.). The isolated yield of perfluoro-tert-butyl trifluoroacetate was 345 g (yield of 55%), and the achieved purity was >98.5% (GC).

### Example 4

### Electrofluorination (ECF) of Trifluoroacetic Acid Tert-Butyl Ester in an Electrochemical Microreactor System (Continuous Process) to Yield Perfluoro-Tert-Butyl Trifluoroacetate.

Fraunhofer is a leader in the field of electrosynthesis in electrochemical microreactors; see, for example, description in:
IMM-Production_Of_Chemicals_By_Electrochemical_Microreactors.pdf (fraunhofer.de).

Some electrochemical reactions in flow chemistry involving amine×HF as fluorine (“F”) source are recently published by Bethan Winterson et al. in Chem. Sci., 2021, 12, 9053.

Commercial systems for electrochemistry in flow chemistry are commercially offered by Vapourtec UK (www.vapourtec.com).

The plastic parts made out of PEEK (polyether ether ketone) used in Vapourtec's reactor (“Ion Integrated”; see Ion integrated electrochemical reactor—Vapourtec) were exchanged by HDPTFE (high density polytetrafluoroethylene) before usage.

The electrodes were out of Nickel (Ni), the reactor volume was 1.2 ml. The pressure resistance of the cell of 5 bar abs. is some limitation. As a consequence the pressure was set to 3 bar abs. by a pressure valve placed after the reactor.

An amount of 90 g (0.53 mol) trifluoroacetic acid tert-butyl ester was fed over a Bronkhorst mass flow controller together with 143.1 g (7.15 mol) HF (containing 5 g of dissolved spray dried KF) over another Bronkhorst mass flow controller over 60 min. continuously into the Vapourtec reactor which was kept at a temperature of −10° C.

The power supply controller of the Vapourtec reactor was set to a current of 4 A and voltage of 36 V.

The product material obtained out of the electrolysis cell was collected in a cooling trap which was also kept at a temperature of −10° C.

The obtained slightly brown colored liquid collected in the cooling trap was quenched into ice water, the organic phase separated off and after drying over Na₂SO₄ it was distilled.

The perfluoro-tert-butyl trifluoroacetate product formed in this Example was distilled off using a 20 cm Vigreux column at 1 bar abs. (transition temperature of 93° C.) leaving behind some only partially fluorinated compounds at the bottom of the distillation apparatus.

The isolated yield of perfluoro-tert-butyl trifluoroacetate was 162 g (yield of 92%), and the achieved purity was >99.0% (GC).

### Example 5

Deprotection of Perfluoro-Tert-Butyl Trifluoroacetate with NaOH.

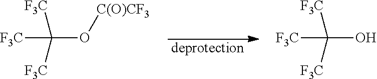

An amount of 100 g (0.3 mol) perfluoro-tert-butyl trifluoroacetate was treated with 50 ml of a saturated NaOH solution in water, and was stirred for 5 h at room temperature (ambient temperature) to saponify to perfluoro-t-BuOH. Afterwards it was extracted three times with 40 ml CH₂Cl₂ (dichloromethane) to obtain perfluoro-t-BuOH in organic phases.

The targeted product perfluoro-t-BuOH was distilled out of the extracted organic phases at 1 bar abs. (transition temperature of 45° C. using a 30 cm Vigreux column after the CH₂Cl₂ as pre-run distillate.

The isolated yield of perfluoro-t-BuOH was 89%, and the purity 99.9% (GC).

The residue (water phase after the extraction) containing TFA (trifluoroacetic acid) and water was acidified with HCl-gas (taken out of a HCl-gas cylinder), and TFA was recycled as trifluoroacetic acid/water azeotrope in ratio of 80:20 with a boiling point of 104° C.

In another trial, extraction of perfluoro-t-BuOH was performed differently. In this alternative extraction 1,1,1,3,3-pentafluorobutane (365mfc) was used as more environmentally friendly extraction agent (as compared to CH₂Cl₂, dichloromethane). The extraction gave an isolated yield of 91% of perfluoro-t-BuOH. The structure of perfluoro-t-BuOH and its purity were confirmed by GC/GC-MS and ¹H-NMR in CDCl₃ (□=3.55 vs. TMS).

### Example 6

Alternative deprotection of perfluoro-tert-butyl trifluoroacetate with 10 wt % amine in water.

An amount of 50 g (0.15 mol) perfluoro-tert-butyl trifluoroacetate was dropped with a dropping funnel into a 10:90 vol-% water solution of NEt₃ in H₂O at 90° C. in a related art stirred glass flask. After 15 min, perfluoro-t-BuOH was detected at the installed reflux condenser and isolated as colorless liquid with boiling point of 44.5° C. The isolated yield of perfluoro-t-BuOH was 97%.

### Example 7

### Trifluoroacetic Acid Anhydride (TFAH) as Protecting Group Raw Material in a Coil Reactor (Continuous Process).

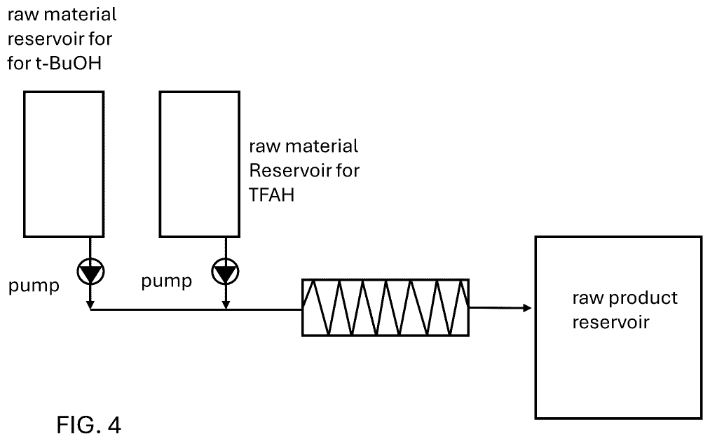

See FIG. 4 for scheme of apparatus and process.

This reaction is described in Tetrahedron Letters (2002), 43(48), 8687-8691 but with standard equipment. In this trial according to the disclosure, a coil reactor was used.

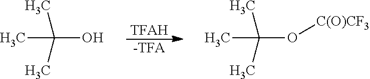

Apparatus: A coil reactor made out of Hastelloy C4 (1 m length, diameter: 0.5 cm) (same as in Example 1), a raw material reservoir (11) with the TFAH raw material, another raw material cylinder with the t-BuOH, both raw materials connected each with a piston pump to the coil reactor. Both feeds are equipped with Bronkhorst mass flow controllers, raw product reservoir (raw product trap) is equipped with a pressure valve set to 2 bar abs., going into a basic scrubber; but in contrast to Example 1, no material is leaving over that valve as trifluoroacetic acid (TFA) is formed in equimolar amounts instead of HCl.

Raw materials: TFAH (from Sigma Aldrich Taufkirchen) and t-BuOH (technical grade)

In the coil reactor apparatus above, 96.4 g (1.3 mol) t-BuOH from Aldrich were placed into the raw material reservoir and the dosage pump started with a feed of 1.3 mol/h. Right after start of the pump, TFAH (273.0 g/h; 1.3 mol/h) was also fed into the coil reactor. The reactor was kept with a simple water bath at 30° C., pressure valve set to 2 bar abs.). An exothermic reaction could be observed immediately, the material after the coil reactor was collected in the raw material reservoir kept at 0° C. by external cooling. After 1 h all the feeds were stopped. The pressure of raw material reservoir was released to atmospheric pressure. A sample taken for GC analysis indicated a quantitative conversion of t-BuOH to trifluoroacetic acid tert-butyl ester which was purified by distillation.

### Example 8

### Continuous Preparation of Tert-Butyl Trifluorosulfonate in a Coil Reactor.

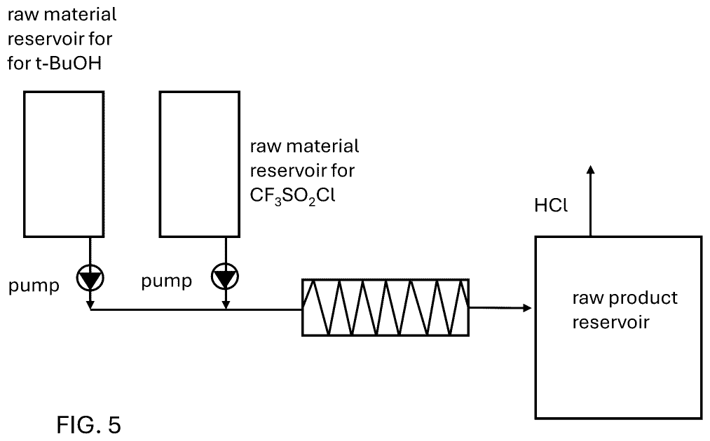

See FIG. 5 for scheme of apparatus and process.

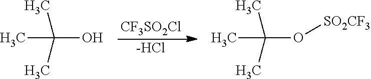

Apparatus: Same equipment as in example 7. Both feeds equipped with Bronkhorst mass flow controllers, raw product reservoir is equipped with a pressure valve going into a basic scrubber for allowing the formed HCl to leave.

Raw materials: Triflyl chloride (product No 164798) and t-BuOH from Sigma Aldrich Taufkirchen.

In the coil reactor apparatus above, 96.4 g (1.3 mol) t-BuOH from Aldrich were placed into the raw material reservoir and the dosage pump started with a feed of 1.3 mol/h. Right after start of the pump, 219.1 g (1.3 mol) CF₃SO₂Cl was also fed into the coil reactor. The reactor was kept with a simple water bath at 26° C., pressure valve set to 3 bar abs. An exothermic reaction could be observed immediately, the material after the coil reactor was collected in the raw material reservoir kept at 0° C. by external cooling. After 1 h the raw material tanks were empty and all the feeds were stopped. The pressure of raw material reservoir was released to atmospheric pressure. A sample taken for GC (and GC-MS) analysis indicated a quantitative conversion of t-BuOH to tert-butyl trifluorosulfonate.

### Example 9

### Electrofluorination (ECF) of Tert-Butyl Trifluorosulfonate (Trifluoroacetic Acid Tert-Butyl Ester) in an Electrochemical Microreactor System (Continuous Process) To Yield Perfluoro-Tert-Butyl Trifluoroacetate.

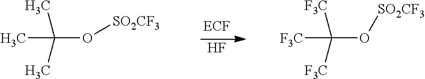

In the same apparatus as used in Example 4, 109.3 g (0.53 mol) tert-butyl trifluorosulfonate was fed over a Bronkhorst mass flow controller together with 143.1 g (7.15 mol) HF (containing 5 g dissolved spray dried KF) over another Bronkhorst mass flow controller over 60 min. continuously into the Vapourtec reactor which was kept at a temperature of −10° C.

The power supply controller of the Vapourtec was set to a current of 4 A and voltage of 32 V.

The product material obtained out of the electrolysis cell was collected in a cooling trap which was also kept at a temperature of −10° C.

The obtained slightly brown colored liquid collected in the trap was quenched into ice water, the organic phase separated off and after drying over Na₂SO₄ it was distilled.

The perfluoro-tert-butyl trifluorosulfonate product formed in this Example was distilled off using a 20 cm Vigreux column at 1 bar abs. (transition temperature of 93° C.) leaving behind some only partially fluorinated compounds at the bottom of the distillation apparatus.

The isolated yield of perfluoro-tert-butyl trifluorosulfonate was 155 g (yield of 79%), and the achieved purity was >99.5% (GC).

### Example 10

### Deprotection of Perfluoro-Tert-Butyl Trifluorosulfonate.

An amount of 55 g (0.15 mol) perfluoro-tert-butyl trifluorosulfonate was topped with a dropping funnel into a 10:90 vol % water solution of NEt₃ in H₂O at 90° C. in a stirred glass flask. After 28 min, perfluoro-t-BuOH was detected at the installed reflux condenser and isolated as colorless liquid with boiling point of 44.5° C. The isolated yield of perfluoro-t-BuOH was 81%.

The foregoing description of the exemplary embodiments of the disclosure illustrates and describes the present invention. Additionally, the disclosure shows and describes only the exemplary embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

All publications, patents and patent applications cited in this specification are herein incorporated by reference, and for any and all purposes, as if each individual publication, patent or patent application were specifically and individually indicated to be incorporated by reference. In the case of inconsistencies, the present disclosure will prevail.

1. A process for the manufacture of a hydroxyl group protected nonafluoro-tert-butyl alcohol ester compound of formula (I), 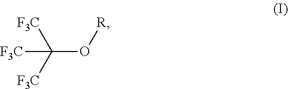
wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CF₂ClSO₂—, CFCl₂SO₂—, CCl₃SO₂—, CF₂HSO₂—, CFH₂SO₂—, CH₃SO₂—, and PfSO₂—, and
wherein P_{f} denotes a partially or perfluorinated C₂-C₄ residue, the method comprising:
reacting a hydroxyl group protected tert-butyl alcohol compound of formula (II), 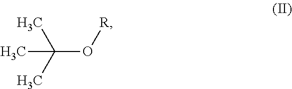
wherein R has a same meaning as above,
in an electrofluorination step in a presence of anhydrous hydrogen fluoride to obtain the hydroxyl group protected nonafluoro-tert-butyl alcohol ester compound of formula (I).

2. The process of claim 1, further comprising:
performing, in parallel and/or subsequent to the electrofluorination step, an isolating and/or purifying step to obtain the isolated and/or purified hydroxyl group protected nonafluoro-tert-butyl alcohol ester compound of formula (I).

3. The process according to claim 1, wherein the hydroxyl group protected nonafluoro-tert-butyl alcohol ester compound of formula (I) is prepared in a protecting reaction step by reacting a tert-butyl alcohol compound of formula (III), 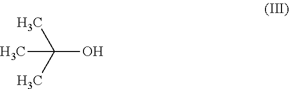
with a hydroxyl group protecting agent of formula R—X (IV), wherein
R has a same meaning as above, and
X denotes a hydrogen atom, a halogen atom, or an —O—R group, each respective R being selected independently,
to obtain the hydroxyl group protected tert-butyl alcohol compound of formula (II).

4. The process according to claim 3, further comprising:
isolating and/or purifying the hydroxyl group protected tert-butyl alcohol compound of formula (II).

5. The process according to claim 1, further comprising:
deprotecting the hydroxyl group protected nonafluoro-tert-butyl alcohol ester compound of formula (I) by removing the hydroxyl group protecting group R to obtain the nonafluoro-tert-butyl alcohol compound of formula (V),

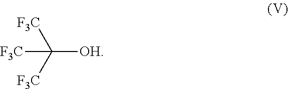 [SMILES: OC(C(F)(F)F)(C(F)(F)F)C(F)(F)F]

6. The process according to claim 1, further comprising:
carrying out the electrofluorination step in a presence of an inorganic halogenide salt.

7. The process according to claim 6, wherein the inorganic halogenide salt is spray dried potassium fluoride.

8. The process according to claim 1, further comprising:
preparing the anhydrous hydrogen fluoride by converting mineral fluorspar (CaF₂) with H₂SO₄ to a mixture essentially including hydrogen fluoride (HF) and CaSO₄;
separating off the anhydrous hydrogen fluoride from the mixture; and
optionally, further purifying the anhydrous hydrogen fluoride.

9. The process according to claim 1, further comprising:
performing the electrofluorination step as a flow electrolysis.

10. The process according to claim 9, wherein the flow electrolysis is performed:
(i) by continuously circulating electrolyte in an electrolysis reactor; or
(ii) as a continuous one time pass-by electrofluorination of starting material at an anode/cathode mix of electrodes in the electrolysis reactor.

11. The process according to claim 1, wherein the electrofluorination step is carried out in an electrochemical microreactor.

12. The process according to claim 11, wherein the electrochemical microreactor is a sandwich microreactor having the anode/cathode mix and a microchannel design, and
wherein the continuous one time pass-by electrofluorination of starting material is performed at the anode/cathode mix of electrodes.

13. The process according to claim 12, wherein the anode of the anode/cathode mix is made from porous nickel material.

14. The process according to claim 1, wherein the electrofluorination is carried out in an electrolysis cell.

15. The process according to claim 1, further comprising:
applying a voltage of 5 to 6 V in the electrofluorination.

16. The process according to claim 1, wherein a current density in the electrofluorination is 0.02 A/cm².

17. The process according to claim 1, wherein the electrofluorination is carried out at a temperature of 0° C. to 20° C.

18. The process according to claim 1, wherein deprotecting is performed by saponification with an aqueous inorganic base.

19. A reaction mixture for an electrolysis reactor, the reaction mixture comprising:
a hydroxyl group protected nonafluoro-tert-butyl alcohol ester compound of formula (I), 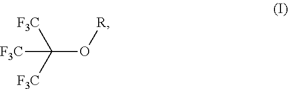
wherein
R denotes a substituent selected from the group consisting of denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CF₂ClSO₂—, CFCl₂SO₂—, CCl₃SO₂—, CF₂HSO₂—, CFH₂SO₂—, CH₃SO₂—, and PfSO₂—, and
wherein P_{f} denotes a partially or perfluorinated C₂-C₄ residue;
anhydrous hydrogen fluoride; and
a spray dried inorganic fluoride salt.

20. A process for the manufacture of a nonafluoro-tert-butyl alcohol compound of formula (V), 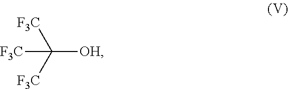 [SMILES: OC(C(F)(F)F)(C(F)(F)F)C(F)(F)F]
the process comprising:
reacting a tert-butyl alcohol compound of formula (III) 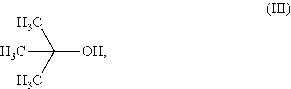
with a hydroxyl group protecting agent R—X of formula (IV),
wherein
R denotes a substituent selected from the group consisting of CF₃CO—, CF₂HCO—, CFH₂CO—, CF₂ClCO—, CFCl₂CO—, CCl₃CO—, CClH₂CO—, CCl₂HCO—, CF₂ClSO₂—, CFCl₂SO₂—, CCl₃SO₂—, CF₂HSO₂—, CFH₂SO₂—, CH₃SO₂—, and PfSO₂—,
wherein P_{f} denotes a partially or perfluorinated C₂-C₄ residue,
wherein X denotes a hydrogen atom, a halogen atom, or an —O—R group,
wherein the R residue of the —O—R group has a same meaning as above,
each respective R being selected independently,
to prepare a nonafluoro-tert-butyl alcohol ester compound of formula (II), 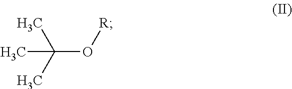
reacting the hydroxyl group protected tert-butyl alcohol compound of formula (II) in a presence of anhydrous hydrogen fluoride in an electrofluorination step to obtain a hydroxyl group protected nonafluoro-tert-butyl alcohol ester compound of formula (I), 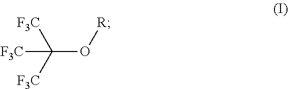
and
deprotecting the hydroxyl group protected nonafluoro-tert-butyl alcohol ester compound of formula (I) by removing the hydroxyl group protecting group R to obtain the nonafluoro-tert-butyl alcohol compound of formula (V).
